SEQUENCE LISTING (170 sequences)

SEQ ID NO: 1 - RNA, 17 nt, Human immunodeficiency virus
auaaagcuugccuugag

SEQ ID NO: 2 - RNA, 17 nt, Human immunodeficiency virus
aggcuaauuuuuuaggg

SEQ ID NO: 3 - RNA, 17 nt, Human immunodeficiency virus
ggcuaauuuuuuaggga

SEQ ID NO: 4 - RNA, 17 nt, Human immunodeficiency virus
gccucaauaaagcuugc

SEQ ID NO: 5 - RNA, 17 nt, Human immunodeficiency virus
uuucggguuuauuacag

SEQ ID NO: 6 - RNA, 17 nt, Human immunodeficiency virus
gcaggacucggcuugcu

SEQ ID NO: 7 - RNA, 17 nt, Human immunodeficiency virus
uggaaaacagauggcag

SEQ ID NO: 8 - RNA, 17 nt, Human immunodeficiency virus
aauaaagcuugccuuga

SEQ ID NO: 9 - RNA, 17 nt, Human immunodeficiency virus
ucucuagcaguggcgcc

SEQ ID NO: 10 - RNA, 17 nt, Human immunodeficiency virus
ggagccaccccacaaga

SEQ ID NO: 11 - RNA, 17 nt, Human immunodeficiency virus
aguggcgcccgaacagg

SEQ ID NO: 12 - RNA, 17 nt, Human immunodeficiency virus
guggcgcccgaacaggg

SEQ ID NO: 13 - RNA, 17 nt, Human immunodeficiency virus
cucgacgcaggacucgg

SEQ ID NO: 14 - RNA, 17 nt, Human immunodeficiency virus
cgcaggacucggcuugc

SEQ ID NO: 15 - RNA, 17 nt, Human immunodeficiency virus
ucaauaaagcuugccuu

SEQ ID NO: 16 - RNA, 17 nt, Human immunodeficiency virus
aggacucggcuugcuga

SEQ ID NO: 17 - RNA, 17 nt, Human immunodeficiency virus
gcaguggcgcccgaaca

SEQ ID NO: 18 - RNA, 17 nt, Human immunodeficiency virus
cucuagcaguggcgccc

SEQ ID NO: 19 - RNA, 17 nt, Human immunodeficiency virus
uagcaguggcgcccgaa

SEQ ID NO: 20 - RNA, 17 nt, Human immunodeficiency virus
agagaugggugcgagag

SEQ ID NO: 21 - RNA, 17 nt, Human immunodeficiency virus
agaugggugcgagagcg

SEQ ID NO: 22 - RNA, 17 nt, Human immunodeficiency virus
cucucgacgcaggacuc

SEQ ID NO: 23 - RNA, 17 nt, Human immunodeficiency virus
uauggaaaacagauggc

SEQ ID NO: 24 - RNA, 17 nt, Human immunodeficiency virus
gaaaacagauggcaggu

SEQ ID NO: 25 - RNA, 17 nt, Human immunodeficiency virus
aagccucaauaaagcuu

SEQ ID NO: 26 - RNA, 17 nt, Human immunodeficiency virus
ggagagagaugggugcg

SEQ ID NO: 27 - RNA, 17 nt, Human immunodeficiency virus
gacgcaggacucggcuu

SEQ ID NO: 28 - RNA, 17 nt, Human immunodeficiency virus
ggaaaacagauggcagg

SEQ ID NO: 29 - RNA, 17 nt, Human immunodeficiency virus
augggugcgagagcguc

SEQ ID NO: 30 - RNA, 17 nt, Human immunodeficiency virus
aaaaggggggauugggg

SEQ ID NO: 31 - RNA, 17 nt, Human immunodeficiency virus
agaaaaggggggauugg

SEQ ID NO: 32 - RNA, 17 nt, Human immunodeficiency virus
gaaaaggggggauuggg

SEQ ID NO: 33 - RNA, 17 nt, Human immunodeficiency virus
ggcuagaaggagagaga

SEQ ID NO: 34 - RNA, 17 nt, Human immunodeficiency virus
uuuuaaaagaaaagggg

SEQ ID NO: 35 - RNA, 17 nt, Human immunodeficiency virus
uauggcaggaagaagcg

SEQ ID NO: 36 - RNA, 17 nt, Human immunodeficiency virus
uggcgcccgaacaggga

SEQ ID NO: 37 - RNA, 17 nt, Human immunodeficiency virus
gagagaugggugcgaga

SEQ ID NO: 38 - RNA, 17 nt, Human immunodeficiency virus
cgacgcaggacucggcu

SEQ ID NO: 39 - RNA, 17 nt, Human immunodeficiency virus
ugacuagcggaggcuag

SEQ ID NO: 40 - RNA, 17 nt, Human immunodeficiency virus
uagaaggagagagaugg

SEQ ID NO: 41 - RNA, 17 nt, Human immunodeficiency virus
aggagagagaugggugc

SEQ ID NO: 42 - RNA, 17 nt, Human immunodeficiency virus
gaaggagagagaugggu

SEQ ID NO: 43 - RNA, 17 nt, Human immunodeficiency virus
ucgacgcaggacucggc

SEQ ID NO: 44 - RNA, 17 nt, Human immunodeficiency virus
cuagcaguggcgcccga

SEQ ID NO: 45 - RNA, 17 nt, Human immunodeficiency virus
gacuagcggaggcuaga

SEQ ID NO: 46 - RNA, 17 nt, Human immunodeficiency virus
gcuagaaggagagagau

SEQ ID NO: 47 - RNA, 17 nt, Human immunodeficiency virus
aaaggggggauuggggg

SEQ ID NO: 48 - RNA, 17 nt, Human immunodeficiency virus
auggaaaacagauggca

SEQ ID NO: 49 - RNA, 17 nt, Human immunodeficiency virus
caggcuaauuuuuuagg

SEQ ID NO: 50 - RNA, 17 nt, Human immunodeficiency virus
cucaauaaagcuugccu

SEQ ID NO: 51 - RNA, 17 nt, Human immunodeficiency virus
caauaaagcuugccuug

SEQ ID NO: 52 - RNA, 17 nt, Human immunodeficiency virus
caguggcgcccgaacag

SEQ ID NO: 53 - RNA, 17 nt, Human immunodeficiency virus
gaggcuagaaggagaga

SEQ ID NO: 54 - RNA, 17 nt, Human immunodeficiency virus
uuaaauaaaauaguaag

SEQ ID NO: 55 - RNA, 17 nt, Human immunodeficiency virus
gaugggugcgagagcgu

SEQ ID NO: 56 - RNA, 17 nt, Human immunodeficiency virus
uuaaaagaaaagggggg

SEQ ID NO: 57 - RNA, 17 nt, Human immunodeficiency virus
uuuaaaagaaaaggggg

SEQ ID NO: 58 - RNA, 17 nt, Human immunodeficiency virus
agccucaauaaagcuug

SEQ ID NO: 59 - RNA, 17 nt, Human immunodeficiency virus
ccucaauaaagcuugcc

SEQ ID NO: 60 - RNA, 17 nt, Human immunodeficiency virus
ggcgcccgaacagggac

SEQ ID NO: 61 - RNA, 17 nt, Human immunodeficiency virus
gagagagaugggugcga

SEQ ID NO: 62 - RNA, 17 nt, Human immunodeficiency virus
agcaguggcgcccgaac

SEQ ID NO: 63 - RNA, 17 nt, Human immunodeficiency virus
auggcaggaagaagcgg

SEQ ID NO: 64 - RNA, 17 nt, Human immunodeficiency virus
aggcuagaaggagagag

SEQ ID NO: 65 - RNA, 17 nt, Human immunodeficiency virus
uaaaagaaaagggggga

SEQ ID NO: 66 - RNA, 17 nt, Human immunodeficiency virus
gagaugggugcgagagc

SEQ ID NO: 67 - RNA, 17 nt, Human immunodeficiency virus
aaaacagauggcaggug

SEQ ID NO: 68 - RNA, 17 nt, Human immunodeficiency virus
acgcaggacucggcuug

SEQ ID NO: 69 - RNA, 17 nt, Human immunodeficiency virus
aaggagagagaugggug

SEQ ID NO: 70 - RNA, 17 nt, Human immunodeficiency virus
cuagaaggagagagaug

SEQ ID NO: 71 - RNA, 17 nt, Human immunodeficiency virus
ucuagcaguggcgcccg

SEQ ID NO: 72 - RNA, 17 nt, Human immunodeficiency virus
uggcaggaagaagcgga

SEQ ID NO: 73 - RNA, 17 nt, Human immunodeficiency virus
agaaggagagagauggg

SEQ ID NO: 74 - RNA, 17 nt, Human immunodeficiency virus
uccuauggcaggaagaa

SEQ ID NO: 75 - RNA, 17 nt, Human immunodeficiency virus
uggaaaggugaaggggc

SEQ ID NO: 76 - RNA, 17 nt, Human immunodeficiency virus
aucucuagcaguggcgc

SEQ ID NO: 77 - RNA, 38 nt, Artificial Sequence
  Description of Artificial Sequence Enzymatic Nucleic Acid
cucaaggccugaugaggccguuaggccgaaagcuuuau

SEQ ID NO: 78 - RNA, 38 nt, Artificial Sequence
  Description of Artificial Sequence Enzymatic Nucleic Acid
cccuaaaacugaugaggccguuaggccgaaauuagccu

SEQ ID NO: 79 - RNA, 38 nt, Artificial Sequence
  Description of Artificial Sequence Enzymatic Nucleic Acid
ucccuaaacugaugaggccguuaggccgaaaauuagcc

SEQ ID NO: 80 - RNA, 38 nt, Artificial Sequence
  Description of Artificial Sequence Enzymatic Nucleic Acid
gcaagcuucugaugaggccguuaggccgaaauugaggc

SEQ ID NO: 81 - RNA, 38 nt, Artificial Sequence
  Description of Artificial Sequence Enzymatic Nucleic Acid
cuguaauacugaugaggccguuaggccgaaacccgaaa

SEQ ID NO: 82 - RNA, 38 nt, Artificial Sequence
  Description of Artificial Sequence Enzymatic Nucleic Acid
agcaagcccugaugaggccguuaggccgaaaguccugc

SEQ ID NO: 83 - RNA, 38 nt, Artificial Sequence
  Description of Artificial Sequence Enzymatic Nucleic Acid
cugccauccugaugaggccguuaggccgaanuuuucca

SEQ ID NO: 84 - RNA, 38 nt, Artificial Sequence
  Description of Artificial Sequence Enzymatic Nucleic Acid
ucaaggcacugaugaggccguuaggccgaancuuuauu

SEQ ID NO: 85 - RNA, 38 nt, Artificial Sequence
  Description of Artificial Sequence Enzymatic Nucleic Acid
ggcgccaccugaugaggccguuaggccgaancuagaga

SEQ ID NO: 86 - RNA, 38 nt, Artificial Sequence
  Description of Artificial Sequence Enzymatic Nucleic Acid
ucuuguggcugaugaggccguuaggccgaanuggcucc

SEQ ID NO: 87 - RNA, 38 nt, Artificial Sequence
  Description of Artificial Sequence Enzymatic Nucleic Acid
ccuguucgcugaugaggccguuaggccgaancgccacu

SEQ ID NO: 88 - RNA, 38 nt, Artificial Sequence
  Description of Artificial Sequence Enzymatic Nucleic Acid
cccuguuccugaugaggccguuaggccgaangcgccac

SEQ ID NO: 89 - RNA, 38 nt, Artificial Sequence
  Description of Artificial Sequence Enzymatic Nucleic Acid
ccgagucccugaugaggccguuaggccgaancgucgag

SEQ ID NO: 90 - RNA, 38 nt, Artificial Sequence
  Description of Artificial Sequence Enzymatic Nucleic Acid
gcaagccgcugaugaggccguuaggccgaanuccugcg

SEQ ID NO: 91 - RNA, 37 nt, Artificial Sequence
  Description of Artificial Sequence Enzymatic Nucleic Acid
aaggcaaggccgaaaggcgagugaggucuuuuauuga

SEQ ID NO: 92 - RNA, 37 nt, Artificial Sequence
  Description of Artificial Sequence Enzymatic Nucleic Acid
ucagcaaggccgaaaggcgagugaggucucgaguccu

SEQ ID NO: 93 - RNA, 37 nt, Artificial Sequence
  Description of Artificial Sequence Enzymatic Nucleic Acid
uguucggggccgaaaggcgagugaggucugccacugc

SEQ ID NO: 94 - RNA, 37 nt, Artificial Sequence
  Description of Artificial Sequence Enzymatic Nucleic Acid
gggcgccagccgaaaggcgagugaggucuugcuagag

SEQ ID NO: 95 - RNA, 37 nt, Artificial Sequence
  Description of Artificial Sequence Enzymatic Nucleic Acid
uucgggcggccgaaaggcgagugaggucucacugcua

SEQ ID NO: 96 - RNA, 37 nt, Artificial Sequence
  Description of Artificial Sequence Enzymatic Nucleic Acid
cucucgcagccgaaaggcgagugaggucuccaucucu

SEQ ID NO: 97 - RNA, 37 nt, Artificial Sequence
  Description of Artificial Sequence Enzymatic Nucleic Acid
cgcucucggccgaaaggcgagugaggucuacccaucu

SEQ ID NO: 98 - RNA, 37 nt, Artificial Sequence
  Description of Artificial Sequence Enzymatic Nucleic Acid
gaguccuggccgaaaggcgagugaggucugucgagag

SEQ ID NO: 99 - DNA, 31 bp, Artificial Sequence
  Description of Artificial Sequence Enzymatic Nucleic Acid
aaggcaagggctagctacaacgatttattga

SEQ ID NO: 100 - DNA, 31 bp, Artificial Sequence
  Description of Artificial Sequence Enzymatic Nucleic Acid
tcagcaagggctagctacaacgacgagtcct

SEQ ID NO: 101 - DNA, 31 bp, Artificial Sequence
  Description of Artificial Sequence Enzymatic Nucleic Acid
tgttcgggggctagctacaacgagccactgc

SEQ ID NO: 102 - DNA, 31 bp, Artificial Sequence
  Description of Artificial Sequence Enzymatic Nucleic Acid
gggcgccaggctagctacaacgatgctagag

SEQ ID NO: 103 - DNA, 31 bp, Artificial Sequence
  Description of Artificial Sequence Enzymatic Nucleic Acid
ttcgggcgggctagctacaacgacactgcta

SEQ ID NO: 104 - DNA, 31 bp, Artificial Sequence
  Description of Artificial Sequence Enzymatic Nucleic Acid
ctctcgcaggctagctacaacgaccatctct

SEQ ID NO: 105 - DNA, 31 bp, Artificial Sequence
  Description of Artificial Sequence Enzymatic Nucleic Acid
cgctctcgggctagctacaacgaacccatct

SEQ ID NO: 106 - DNA, 31 bp, Artificial Sequence
  Description of Artificial Sequence Enzymatic Nucleic Acid
gagtcctgggctagctacaacgagtcgagag

SEQ ID NO: 107 - DNA, 31 bp, Artificial Sequence
  Description of Artificial Sequence Enzymatic Nucleic Acid
gccatctgggctagctacaacgatttccata

SEQ ID NO: 108 - DNA, 31 bp, Artificial Sequence
  Description of Artificial Sequence Enzymatic Nucleic Acid
acctgccaggctagctacaacgactgttttc

SEQ ID NO: 109 - DNA, 31 bp, Artificial Sequence
  Description of Artificial Sequence Enzymatic Nucleic Acid
aagctttaggctagctacaacgatgaggctt

SEQ ID NO: 110 - DNA, 31 bp, Artificial Sequence
  Description of Artificial Sequence Enzymatic Nucleic Acid
cgcacccaggctagctacaacgactctctcc

SEQ ID NO: 111 - DNA, 31 bp, Artificial Sequence
  Description of Artificial Sequence Enzymatic Nucleic Acid
aagccgagggctagctacaacgacctgcgtc

SEQ ID NO: 112 - RNA, 48 nt, Artificial Sequence
  Description of Artificial Sequence Enzymatic Nucleic Acid
aaggcaagggaggaaacucccuucaaggacaucguccggguuuauuga

SEQ ID NO: 113 - RNA, 48 nt, Artificial Sequence
  Description of Artificial Sequence Enzymatic Nucleic Acid
ucagcaagggaggaaacucccuucaaggacaucguccgggcgaguccu

SEQ ID NO: 114 - RNA, 48 nt, Artificial Sequence
  Description of Artificial Sequence Enzymatic Nucleic Acid
uguucgggggaggaaacucccuucaaggacaucguccggggccacugc

SEQ ID NO: 115 - RNA, 48 nt, Artificial Sequence
  Description of Artificial Sequence Enzymatic Nucleic Acid
gggcgccaggaggaaacucccuucaaggacaucguccgggugcuagag

SEQ ID NO: 116 - RNA, 48 nt, Artificial Sequence
  Description of Artificial Sequence Enzymatic Nucleic Acid
uucgggcgggaggaaacucccuucaaggacaucguccgggcacugcua

SEQ ID NO: 117 - RNA, 48 nt, Artificial Sequence
  Description of Artificial Sequence Enzymatic Nucleic Acid
cucucgcaggaggaaacucccuucaaggacaucguccgggccaucucu

SEQ ID NO: 118 - RNA, 48 nt, Artificial Sequence
  Description of Artificial Sequence Enzymatic Nucleic Acid
cgcucucgggaggaaacucccuucaaggacaucguccgggacccaucu

SEQ ID NO: 119 - RNA, 48 nt, Artificial Sequence
  Description of Artificial Sequence Enzymatic Nucleic Acid
gaguccugggaggaaacucccuucaaggacaucguccggggucgagag

SEQ ID NO: 120 - RNA, 48 nt, Artificial Sequence
  Description of Artificial Sequence Enzymatic Nucleic Acid
ccugccauggaggaaacucccuucaaggacaucguccggguguuuucc

SEQ ID NO: 121 - RNA, 48 nt, Artificial Sequence
  Description of Artificial Sequence Enzymatic Nucleic Acid
gacgcucuggaggaaacucccuucaaggacaucguccggggcacccau

SEQ ID NO: 122 - RNA, 48 nt, Artificial Sequence
  Description of Artificial Sequence Enzymatic Nucleic Acid
ccccaaucggaggaaacucccuucaaggacaucguccgggccccuuuu

SEQ ID NO: 123 - RNA, 48 nt, Artificial Sequence
  Description of Artificial Sequence Enzymatic Nucleic Acid
ccaaucccggaggaaacucccuucaaggacaucguccgggccuuuucu

SEQ ID NO: 124 - RNA, 48 nt, Artificial Sequence
  Description of Artificial Sequence Enzymatic Nucleic Acid
cccaauccggaggaaacucccuucaaggacaucguccgggcccuuuuc

SEQ ID NO: 125 - RNA, 48 nt, Artificial Sequence
  Description of Artificial Sequence Enzymatic Nucleic Acid
ucucucucggaggaaacucccuucaaggacaucguccggguucuagcc

SEQ ID NO: 126 - RNA, 48 nt, Artificial Sequence
  Description of Artificial Sequence Enzymatic Nucleic Acid
ccccuuuuggaggaaacucccuucaaggacaucguccggguuuuaaaa

SEQ ID NO: 127 - RNA, 48 nt, Artificial Sequence
  Description of Artificial Sequence Enzymatic Nucleic Acid
cgcuucuuggaggaaacucccuucaaggacaucguccgggcugccaua

SEQ ID NO: 128 - RNA, 48 nt, Artificial Sequence
  Description of Artificial Sequence Enzymatic Nucleic Acid
ucccuguuggaggaaacucccuucaaggacaucguccggggggcgcca

SEQ ID NO: 129 - RNA, 48 nt, Artificial Sequence
  Description of Artificial Sequence Enzymatic Nucleic Acid
ucucgcacggaggaaacucccuucaaggacaucguccgggcaucucuc

SEQ ID NO: 130 - RNA, 48 nt, Artificial Sequence
  Description of Artificial Sequence Enzymatic Nucleic Acid
agccgaguggaggaaacucccuucaaggacaucguccgggcugcgucg

SEQ ID NO: 131 - RNA, 48 nt, Artificial Sequence
  Description of Artificial Sequence Enzymatic Nucleic Acid
cuagccucggaggaaacucccuucaaggacaucguccggggcuaguca

SEQ ID NO: 132 - RNA, 48 nt, Artificial Sequence
  Description of Artificial Sequence Enzymatic Nucleic Acid
ccaucucuggaggaaacucccuucaaggacaucguccggguccuucua

SEQ ID NO: 133 - RNA, 48 nt, Artificial Sequence
  Description of Artificial Sequence Enzymatic Nucleic Acid
gcacccauggaggaaacucccuucaaggacaucguccgggucucuccu

SEQ ID NO: 134 - RNA, 48 nt, Artificial Sequence
  Description of Artificial Sequence Enzymatic Nucleic Acid
acccaucuggaggaaacucccuucaaggacaucguccgggucuccuuc

SEQ ID NO: 135 - RNA, 48 nt, Artificial Sequence
  Description of Artificial Sequence Enzymatic Nucleic Acid
gccgagucggaggaaacucccuucaaggacaucguccgggugcgucga

SEQ ID NO: 136 - RNA, 48 nt, Artificial Sequence
  Description of Artificial Sequence Enzymatic Nucleic Acid
ucgggcgcggaggaaacucccuucaaggacaucguccgggacugcuag

SEQ ID NO: 137 - RNA, 48 nt, Artificial Sequence
  Description of Artificial Sequence Enzymatic Nucleic Acid
ucuagccuggaggaaacucccuucaaggacaucguccgggcgcuaguc

SEQ ID NO: 138 - RNA, 48 nt, Artificial Sequence
  Description of Artificial Sequence Enzymatic Nucleic Acid
aucucucuggaggaaacucccuucaaggacaucguccgggcuucuagc

SEQ ID NO: 139 - RNA, 48 nt, Artificial Sequence
  Description of Artificial Sequence Enzymatic Nucleic Acid
cccccaauggaggaaacucccuucaaggacaucguccgggcccccuuu

SEQ ID NO: 140 - RNA, 15 nt, Human immunodeficiency virus
gagaugggugcgaga

SEQ ID NO: 141 - RNA, 15 nt, Human immunodeficiency virus
auggaaaacagaugg

SEQ ID NO: 142 - RNA, 15 nt, Human immunodeficiency virus
aaaacagauggcagg

SEQ ID NO: 143 - RNA, 15 nt, Human immunodeficiency virus
agccucaauaaagcu

SEQ ID NO: 144 - RNA, 15 nt, Human immunodeficiency virus
gagagagaugggugc

SEQ ID NO: 145 - RNA, 15 nt, Human immunodeficiency virus
caauaaagcuugccu

SEQ ID NO: 146 - RNA, 15 nt, Human immunodeficiency virus
ggacucggcuugcug

SEQ ID NO: 147 - RNA, 15 nt, Human immunodeficiency virus
gaugggugcgagagc

SEQ ID NO: 148 - RNA, 15 nt, Human immunodeficiency virus
ucucgacgcaggacu

SEQ ID NO: 149 - DNA, 29 bp, Artificial Sequence
  Description of Artificial Sequence Enzymatic Nucleic Acid
ucucgcaggctagctacaacgaccaucuc

SEQ ID NO: 150 - DNA, 29 bp, Artificial Sequence
  Description of Artificial Sequence Enzymatic Nucleic Acid
ccaucugggctagctacaacgauuuccau

SEQ ID NO: 151 - DNA, 29 bp, Artificial Sequence
  Description of Artificial Sequence Enzymatic Nucleic Acid
ccugccaggctagctacaacgacuguuuu

SEQ ID NO: 152 - DNA, 29 bp, Artificial Sequence
  Description of Artificial Sequence Enzymatic Nucleic Acid
agcuuuaggctagctacaacgaugaggcu

SEQ ID NO: 153 - DNA, 29 bp, Artificial Sequence
  Description of Artificial Sequence Enzymatic Nucleic Acid
gcacccaggctagctacaacgacucucuc

SEQ ID NO: 154 - RNA, 35 nt, Artificial Sequence
  Description of Artificial Sequence Enzymatic Nucleic Acid
aggcaaggccgaaaggcgagugaggucuuuuauug

SEQ ID NO: 155 - RNA, 35 nt, Artificial Sequence
  Description of Artificial Sequence Enzymatic Nucleic Acid
cagcaaggccgaaaggcgagugaggucucgagucc

SEQ ID NO: 156 - RNA, 35 nt, Artificial Sequence
  Description of Artificial Sequence Enzymatic Nucleic Acid
ucucgcagccgaaaggcgagugaggucuccaucuc

SEQ ID NO: 157 - RNA, 35 nt, Artificial Sequence
  Description of Artificial Sequence Enzymatic Nucleic Acid
gcucucggccgaaaggcgagugaggucuacccauc

SEQ ID NO: 158 - RNA, 35 nt, Artificial Sequence
  Description of Artificial Sequence Enzymatic Nucleic Acid
aguccuggccgaaaggcgagugaggucugucgaga

SEQ ID NO: 159 - RNA, 10 nt, Artificial Sequence
  Description of Artificial Sequence Exemplary Stem II Sequence
gccguuaggc

SEQ ID NO: 160 - RNA, 15 nt, Artificial Sequence
  Description of Artificial Sequence Generic substrate sequence
nnnnnnuhnnnnnnn

SEQ ID NO: 161 - RNA, 36 nt, Artificial Sequence
  Description of Artificial Sequence Enzymatic Nucleic Acid
nnnnnnncugaugagnnngaaannncgaaannnnnn

SEQ ID NO: 162 - RNA, 14 nt, Artificial Sequence
  Description of Artificial Sequence Generic substrate sequence
nnnnnchnnnnnnn

SEQ ID NO: 163 - RNA, 35 nt, Artificial Sequence
  Description of Artificial Sequence Enzymatic Nucleic Acid
nnnnnnncugaugagnnngaaannncgaannnnnn

SEQ ID NO: 164 - RNA, 15 nt, Artificial Sequence
  Description of Artificial Sequence Generic substrate sequence
nnnnnnygnnnnnnn

SEQ ID NO: 165 - RNA, 35 nt, Artificial Sequence
  Description of Artificial Sequence Enzymatic Nucleic Acid
nnnnnnnugauggcaugcacuaugcgcgnnnnnnn

SEQ ID NO: 166 - RNA, 48 nt, Artificial Sequence
  Description of Artificial Sequence Enzymatic Nucleic Acid
gugugcaaccggaggaaacucccuucaaggacgaaaguccgggacggg

SEQ ID NO: 167 - RNA, 16 nt, Artificial Sequence
  Description of Artificial Sequence Substrate sequence
gccguggguugcacac

SEQ ID NO: 168 - RNA, 36 nt, Artificial Sequence
  Description of Artificial Sequence Enzymatic Nucleic Acid
gugccuggccgaaaggcgagugaggucugccgcgcn

SEQ ID NO: 169 - RNA, 15 nt, Artificial Sequence
  Description of Artificial Sequence Substrate sequence
gcgcggcgcaggcac

SEQ ID NO: 170 - DNA, 16 bp, Artificial Sequence
  Description of Artificial Sequence Enzymatic Nucleic Acid Motif
rggctagctacaacga

(19) United States
(12) Patent Application Publication
(10) Pub. No.: US 2003/0124513 A1
(43) Pub. Date: Jul. 3, 2003

(54) Enzymatic nucleic acid treatment of diseases or conditions related to levels of HIV

(76) Inventor: James McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/157,580

(22) Filed: May 29, 2002

Related U.S. Application Data
(60) Provisional application No. 60/294,140, filed on May 29, 2001.

(51) Int. Cl. A61K 48/00; C12Q 1/70; C07H 21/02

(52) U.S. Cl. 435/5; 514/44; 536/23.1

(57) ABSTRACT

The present invention relates to nucleic acid molecules, including enzymatic nucleic acid molecules, such as hammerhead ribozymes, DNAzymes, siRNA, aptamers, decoys and allozymes, which modulate the expression of HIV genes.

This patent application claims priority from U.S. Ser. No. 60/294,140, filed May 29, 2001, entitled “ENZYMATIC NUCLEIC ACID TREATMENT OF DISEASES OR CONDITIONS RELATED TO LEVELS OF HIV.” This application is hereby incorporated by reference herein in its entirety including the drawings.

### FIELD OF THE INVENTION

The present invention relates to therapeutic compositions and methods for the treatment or diagnosis of diseases or conditions related to Human Immunodeficiency Virus (HIV) infection, in particular, HIV-1 infection.

### BACKGROUND OF THE INVENTION

Acquired immunodeficiency syndrome (AIDS) is thought to be caused by infection with the human immunodeficiency virus, for example HIV-1. Draper et al., U.S. Pat. Nos. 6,159,692, 5,972,704, 5,693,535, and International PCT Publication Nos. WO WO 93/23569, WO 95/04818, describe enzymatic nucleic acid molecules targeting HIV. Todd et al., International PCT Publication No. WO 99/50452, describe methods for using specific DNAzyme motifs for detecting the presence of certain HIV RNAs. Sriram and Banerjea, 2000, Biochem J., 352, 667-673, describe specific RNA cleaving DNA enzymes targeting HIV-1. Zhang et al., 1999, FEBS Lett., 458, 151-156, describe specific RNA cleaving DNA enzymes used in the inhibition of HIV-1 infection.

### SUMMARY OF THE INVENTION

The present invention features an enzymatic nucleic acid molecule which down regulates expression of a nucleic acid molecule encoding a human immunodeficiency virus (HIV), for example HIV-1, HIV-2, and related viruses such as FIV-1 and SIV-1, or a HIV gene, for example LTR, nef, vif, tat, or rev, wherein the enzymatic nucleic acid molecule comprises a DNAzyme configuration.

The invention also features an enzymatic nucleic acid molecule which down regulates expression of a nucleic acid molecule encoding HIV or a component of HIV such as net, vif, tat, or rev, wherein the enzymatic nucleic acid molecule is in a Inozyme, G-cleaver, Zinzyme, DNAzyme or Amberzyme configuration.

In addition, the present invention features a siRNA nucleic acid molecule which down regulates expression of a nucleic acid molecule encoding a human immunodeficiency virus (HIV), for example HIV-1, HIV-2, and related viruses such as FIV-1 and SIV-1, or a HIV gene, for example LTR, nef, vif, tat, or rev.

The present invention features an enzymatic nucleic acid molecule comprising a sequence selected from the group consisting of SEQ ID NOs. 77-139 and 149-158. The invention also features an enzymatic nucleic acid molecule comprising at least one binding arm wherein one or more of said binding arms comprises a sequence complementary to a sequence selected from the group consisting of SEQ ID NOs. 1-76 and 140-148. In addition, the present invention features a siRNA nucleic acid molecule comprising sequence complementary to a sequence selected from the group consisting of SEQ ID NOs. 1-76 and 140-148.

In another embodiment, the siRNA molecule of the invention has RNA interference activity to HIV-1 expression and/or replication.

In one embodiment, a siRNA molecule of the invention comprises a double stranded RNA wherein one strand of the RNA is complementary to the RNA of HIV-1 genome or genes. In another embodiment, a siRNA molecule of the invention comprises a double stranded RNA wherein one strand of the RNA comprises a portion of a sequence of HIV-1 genome or gene sequence. In yet another embodiment, a siRNA molecule of the invention comprises a double stranded RNA wherein both strands of RNA are connected by a non-nucleotide linker. Alternately, a siRNA molecule of the invention comprises a double stranded RNA wherein both strands of RNA are connected by a nucleotide linker, such as a loop or stem loop structure.

In one embodiment, a single strand component of a siRNA molecule of the invention is from about 14 to about 50 nucleotides in length. In another embodiment, a single strand component of a siRNA molecule of the invention is about 14, 15, 16, 17, 18, 19, 20, 21, 22, 23, 24, 25, 26, 27, or 28 nucleotides in length. In yet another embodiment, a single strand component of a siRNA molecule of the invention is about 23 nucleotides in length. In one embodiment, a siRNA molecule of the invention is from about 28 to about 56 nucleotides in length. In another embodiment, a siRNA molecule of the invention is about 40, 41, 42, 43, 44, 45, 46, 47, 48, 49, 50, 51, or 52 nucleotides in length. In yet another embodiment, a siRNA molecule of the invention is about 46 nucleotides in length.

In one embodiment, a nucleic acid molecule of the invention is adapted to HIV infection or acquired immunodeficiency syndrome (AIDS).

In another embodiment, the enzymatic nucleic acid molecule of the invention has an endonuclease activity to cleave nucleic acid molecules, such as RNA, having HIV sequence.

In yet another embodiment, the enzymatic nucleic acid molecule of the invention is in an Inozyme, Zinzyme, G-cleaver, Amberzyme, DNAzyme or Hammerhead configuration.

In another embodiment, the Inozyme of the invention comprises a sequence complementary to a sequence selected from the group consisting of SEQ ID NOs. 7-14, or comprises a sequence selected from the group consisting of SEQ ID NOs. 83-90.

In another embodiment, the Zinzyme of the invention comprises a sequence complementary to a sequence selected from the group consisting of SEQ ID NOs. 15-22 and 145-148, or comprises a sequence selected from the group consisting of SEQ ID NOs 91-98 and 154-158.

In another embodiment, the Amberzyme of the invention comprises a sequence complementary to a sequence selected from the group consisting of SEQ ID NOs. 15-22 and 28-47, or comprises a sequence selected from the group consisting of SEQ ID NOs 112-139.

In another embodiment, the DNAzyme of the invention comprises a sequence complementary to a sequence selected from the group consisting of SEQ ID NOs. 15-27 and 140-144, or comprises a sequence selected from the group consisting of SEQ ID NOs 99-111 and 149-153.

In another embodiment, the Hammerhead of the invention comprises a sequence complementary to a sequence selected from the group consisting of SEQ ID NOs. 1-6, or comprises a sequence selected from the group consisting of SEQ ID NOs 77-82.

In one embodiment, a nucleic acid molecule of the invention comprises between 12 and 100 bases complementary to a RNA sequence encoding HIV genome, RNA, and/or proteins. In another embodiment, a nucleic acid molecule of the invention comprises between 14 and 24 bases complementary to a RNA sequence encoding HIV genome, RNA, and/or proteins.

In yet another embodiment, a nucleic acid molecule of the invention is chemically synthesized. A nucleic acid molecule of the invention can comprise at least one 2′-sugar modification, at least one nucleic acid base modification, and/or at least one phosphate backbone modification.

The present invention features a mammalian cell including a nucleic acid molecule of the invention. In one embodiment, the mammalian cell of the invention is a human cell.

The invention features a method of reducing HIV activity in a cell, comprising contacting the cell with a nucleic acid molecule of the invention, under conditions suitable for the reduction of HIV activity.

The invention also features a method of treating a subject having a condition associated with the level of HIV, comprising contacting cells of the subject with a nucleic acid molecule of the invention, under conditions suitable for the treatment.

In one embodiment, methods of treatment contemplated by the invention comprise the use of one or more drug therapies under conditions suitable for the treatment.

The invention features a method of cleaving RNA comprising a HIV nucleic acid sequence comprising contacting an enzymatic nucleic acid molecule of the invention with the RNA under conditions suitable for the cleavage. In one embodiment, the cleavage contemplated by the invention is carried out in the presence of a divalent cation, for example Mg²⁺.

In another embodiment, the nucleic acid molecule of the invention comprises a cap structure, wherein the cap structure is at the 5′-end, 3′-end, or both the 5′-end and the 3′-end of the nucleic acid molecule, for example a 3′,3′-linked or 5′,5′-linked deoxyabasic ribose derivative.

The present invention features an expression vector comprising a nucleic acid sequence encoding at least one nucleic acid molecule of the invention in a manner which allows expression of the nucleic acid molecule.

The invention also features a mammalian cell, for example a human cell, including an expression vector contemplated by the invention.

In one embodiment, an expression vector of the invention comprises a nucleic acid sequence encoding two or more nucleic acid molecules, which may be the same or different.

The present invention features a method for treatment of acquired immunodeficiency syndrome (AIDS) or an AIDS related condition, for example Kaposi's sarcoma, lymphoma, cervical cancer, squamous cell carcinoma, cardiac myopathy, rheumatic disease, or opportunistic infection, comprising administering to a subject a nucleic acid molecule of the invention under conditions suitable for the treatment.

In one embodiment, an enzymatic nucleic acid molecule of the invention comprises at least five ribose residues, at least ten 2′-O-methyl modifications, and a 3′-end modification, for example a 3′-3′ inverted abasic moiety.

In another embodiment, an enzymatic nucleic acid molecule of the invention further comprises phosphorothioate linkages on at least three of the 5′ terminal nucleotides.

In yet another embodiment, a DNAzyme of the invention comprises at least ten 2′-O-methyl modifications and a 3′-end modification, for example a 3′-3′ inverted abasic moiety. In a further embodiment, the DNAzyme of the invention further comprises phosphorothioate linkages on at least three of the 5′ terminal nucleotides.

In another embodiment, other drug therapies of the invention comprise antiviral therapy, monoclonal antibody therapy, chemotherapy, radiation therapy, analgesic therapy, or anti-inflammatory therapy.

In yet another embodiment, antiviral therapy of the invention comprises treatment with AZT, ddC, ddI, d4T, 3TC, Ribavirin, delvaridine, nevirapine, efravirenz, ritonavir, saquinivir, indinavir, amprenivir, nelfinavir, or lopinavir.

The invention features a composition comprising an enzymatic nucleic acid molecule of the invention in a pharmaceutically acceptable carrier.

In one embodiment, the invention features a method of administering to a cell, for example a mammalian cell or human cell, a nucleic acid molecule of the invention comprising contacting the cell with the enzymatic nucleic acid molecule under conditions suitable for the administration. The method of administration can be in the presence of a delivery reagent, for example a lipid, cationic lipid, phospholipid, or liposome.

### DETAILED DESCRIPTION OF THE INVENTION

First the drawings will be described briefly.

Drawings

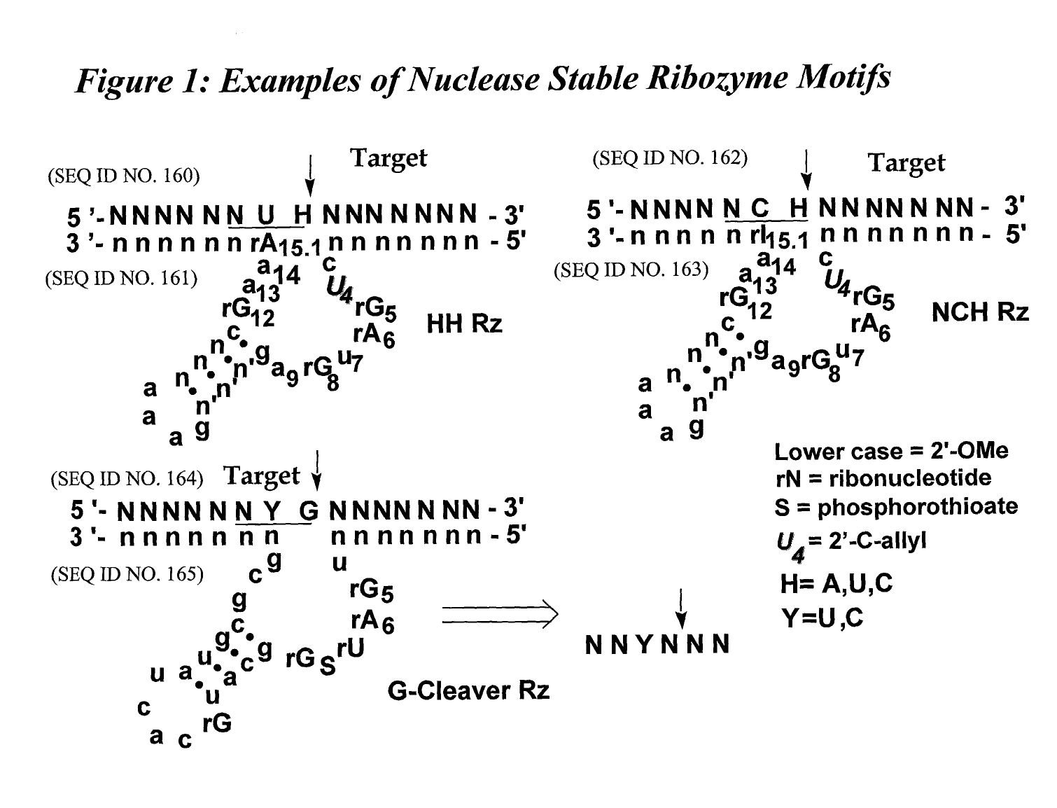

FIG. 1 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120, Eckstein et al., International PCT publication No. WO 99/16871). N or n, represent independently a nucleotide which can be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

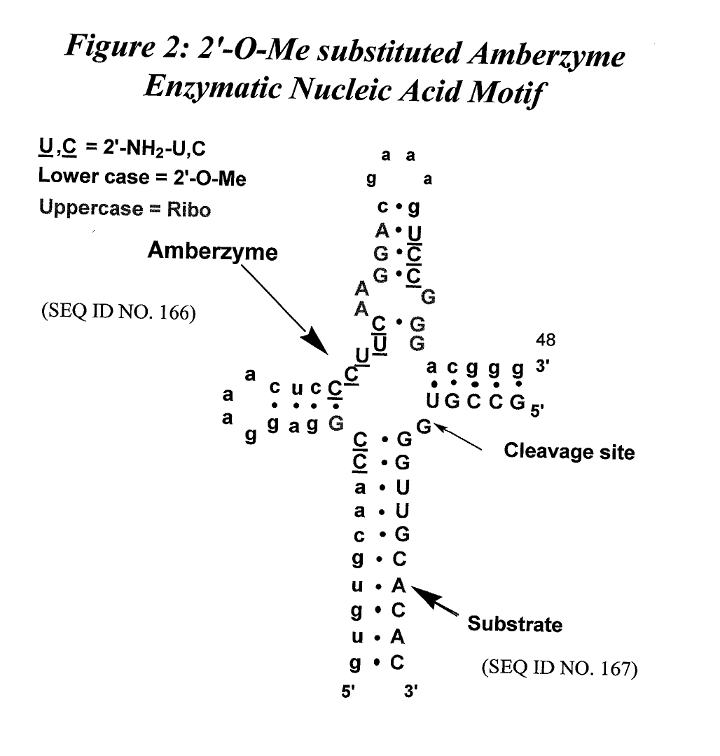

FIG. 2 shows an example of the Amberzyme ribozyme motif that is chemically stabilized (see for example Beigelman et al., International PCT publication No. WO 99/55857).

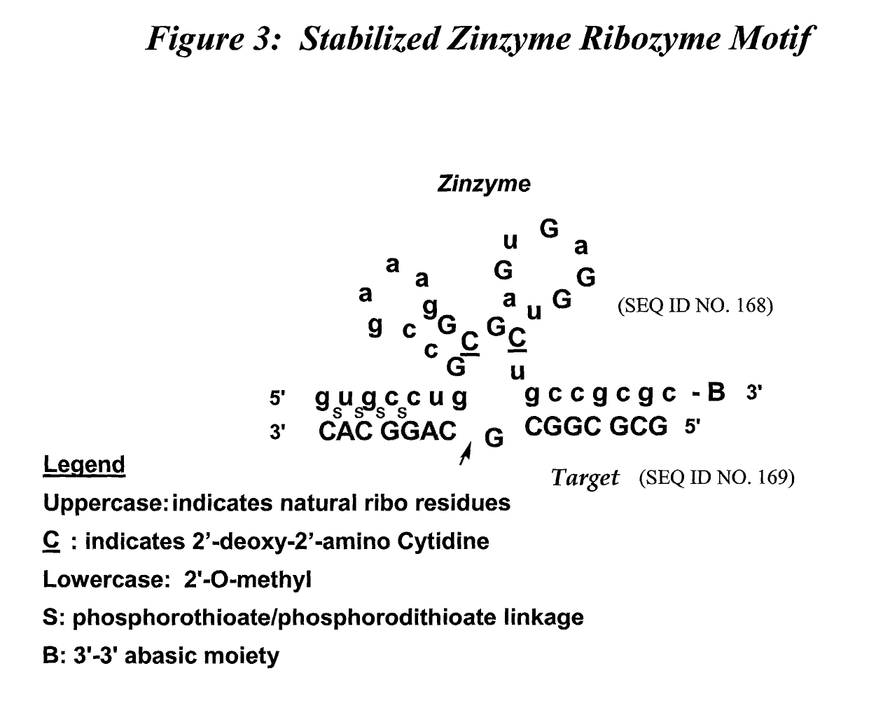

FIG. 3 shows an example of the Zinzyme A ribozyme motif that is chemically stabilized (see for example Beigelman et al., Beigelman et al., International PCT publication No. WO 99/55857).

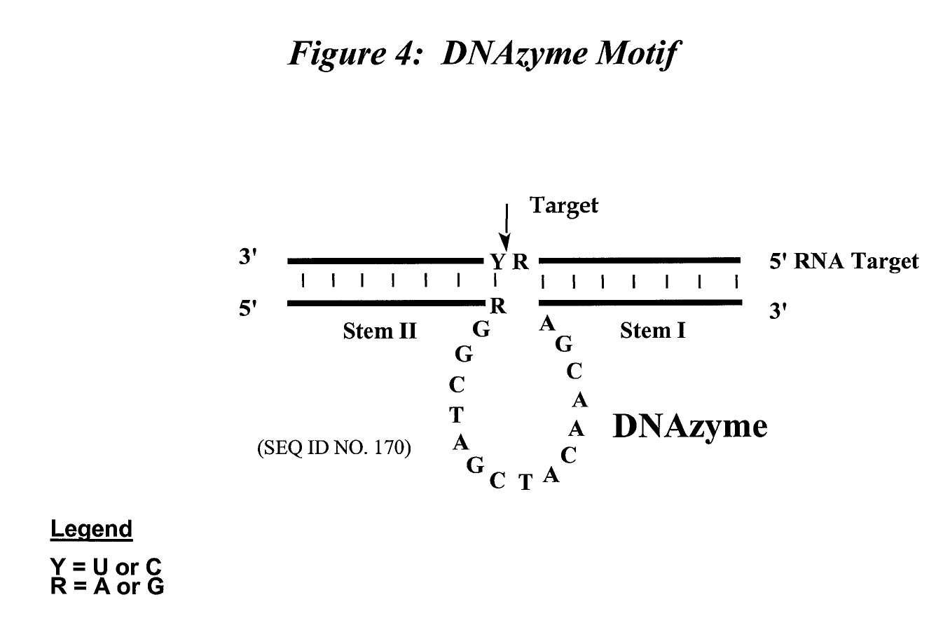

FIG. 4 shows an example of a DNAzyme motif described by Santoro et al., 1997, PNAS, 94, 4262.

The invention features novel enzymatic nucleic acid molecules, siRNA molecules, and methods to modulate gene expression, for example, genes encoding human immunodeficiency virus (HIV), for example HIV-1, HIV-2, and related viruses such as FIV-1 and SIV-1, or a HIV gene, for example LTR, nef, vif, tat, or rev. In particular, the instant invention features nucleic-acid based molecules and methods to inhibit the replication of a HIV or related virus.

The invention features one or more nucleic acid-based molecules and methods that independently or in combination modulate the expression of gene(s) encoding HIV and/or inhibit the replication of HIV. In particular embodiments, the invention features nucleic acid-based molecules and methods that modulate the expression of HIV-1 encoded genes, for example (Genbank Accession No. AJ302647); HIV-2 gene, for example (Genbank Accession No. NC_{—}001722), FIV-1, for example (Genbank Accession No. NC_{—}001482), SIV-1, for example (Genbank Accession No. M66437), LTR, for example included in (Genbank Accession No. AJ302647), nef, for example included in (Genbank Accession No. AJ302647), vif, for example included in (Genbank Accession No. AJ302647), tat, for example included in (Genbank Accession No. AJ302647), and rev, for example included in (Genbank Accession No. AJ302647).

The description below of the various aspects and embodiments is provided with reference to the exemplary HIV-1 gene, referred to herein as HIV. However, the various aspects and embodiments are also directed to other genes which encode HIV proteins and similar viruses to HIV. Those additional genes can be analyzed for target sites using the methods described for HIV. Thus, the inhibition and the effects of such inhibition of the other genes can be performed as described herein.

Due to the high sequence variability of the HIV genome, selection of nucleic acid molecules for broad therapeutic applications would likely involve the conserved regions of the HIV genome. Specifically, the present invention describes nucleic acid molecules that cleave the conserved regions of the HIV genome. Therefore, one nucleic acid molecule can be designed to cleave all the different isolates of HIV. Nucleic acid molecules designed against conserved regions of various HIV isolates can enable efficient inhibition of HIV replication in diverse subject populations and can ensure the effectiveness of the nucleic acid molecules against HIV quasi species which evolve due to mutations in the non-conserved regions of the HIV genome.

In one embodiment, the invention features the use of an enzymatic nucleic acid molecule, preferably in the hammerhead, NCH, G-cleaver, amberzyme, zinzyme and/or DNAzyme motif, to down-regulate the expression of HIV genes or inhibit the replication of HIV.

By “inhibit” or “down-regulate” it is meant that the expression of the gene, or level of RNAs or equivalent RNAs encoding one or more protein subunits or components, or activity of one or more protein subunits or components, such as HIV protein(s), is reduced below that observed in the absence of the nucleic acid molecules of the invention. In one embodiment, inhibition or down-regulation with enzymatic nucleic acid molecule preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation with an siRNA molecule is preferably below that level observed in the presence of, for example, an oligonucleotide with scrambled sequence or with mismatches. In another embodiment, inhibition or down-regulation of HIV expression and/or activity with the nucleic acid molecule of the instant invention is greater in the presence of the nucleic acid molecule than in its absence.

By “up-regulate” is meant that the expression of the gene, or level of RNAs or equivalent RNAs encoding one or more protein subunits or components, or activity of one or more protein subunits or components, such as HIV protein(s), is greater than that observed in the absence of the nucleic acid molecules of the invention. For example, the expression of a gene, such as HIV gene, can be increased in order to treat, prevent, ameliorate, or modulate a pathological condition caused or exacerbated by an absence or low level of gene expression.

By “modulate” is meant that the expression of the gene, or level of RNAs or equivalent RNAs encoding one or more protein subunits or components, or activity of one or more proteins is up-regulated or down-regulated, such that the expression, level, or activity is greater than or less than that observed in the absence of the nucleic acid molecules of the invention.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

By “enzymatic portion” or “catalytic domain” is meant that portion/region of the enzymatic nucleic acid molecule essential for cleavage of a nucleic acid substrate (for example see FIGS. 1-4).

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-4. That is, these arms contain sequences within a enzymatic nucleic acid which are intended to bring enzymatic nucleic acid and target RNA together through complementary base-pairing interactions. The enzymatic nucleic acid of the invention can have binding arms that are contiguous or non-contiguous and may be of varying lengths. The length of the binding arm(s) are preferably greater than or equal to four nucleotides and of sufficient length to stably interact with the target RNA; preferably 12-100 nucleotides; more preferably 14-24 nucleotides long (see for example Werner and Uhlenbeck, supra; Hamman et al., supra;

Hampel et al., EP0360257; Berzal-Herranz et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “Inozyme” or “NCH” motif or configuration is meant, an enzymatic nucleic acid molecule comprising a motif as is generally described as NCH Rz in FIG. 1 and in Ludwig et al., International PCT Publication No. WO 98/58058 and U.S. patent application Ser. No. 08/878,640. Inozymes possess endonuclease activity to cleave RNA substrates having a cleavage triplet NCH/, where N is a nucleotide, C is cytidine and H is adenosine, uridine or cytidine, and /“/” represents the cleavage site. H is used interchangeably with X. Inozymes can also possess endonuclease activity to cleave RNA substrates having a cleavage triplet NCN/, where N is a nucleotide, C is cytidine, and /“/” represents the cleavage site. “I” in FIG. 1 represents an Inosine nucleotide, preferably a ribo-Inosine or xylo-Inosine nucleoside.

By “G-cleaver” motif or configuration is meant, an enzymatic nucleic acid molecule comprising a motif as is generally described as G-cleaver Rz in FIG. 1 and in Eckstein et al., U.S. Pat. No. 6,127,173. G-cleavers possess endonuclease activity to cleave RNA substrates having a cleavage triplet NYN/, where N is a nucleotide, Y is uridine or cytidine and /“/” represents the cleavage site. G-cleavers can be chemically modified as is generally shown in FIG. 1.

By “amberzyme” motif or configuration is meant, an enzymatic nucleic acid molecule comprising a motif as is generally described in FIG. 2 and in Beigelman et al., International PCT publication No. WO 99/55857 and U.S. patent application Ser. No. 09/476,387. Amberzymes possess endonuclease activity to cleave RNA substrates having a cleavage triplet NG/N, where N is a nucleotide, G is guanosine, and /“/” represents the cleavage site. Amberzymes can be chemically modified to increase nuclease stability through substitutions as are generally shown in FIG. 2. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

By “zinzyme” motif or configuration is meant, an enzymatic nucleic acid molecule comprising a motif as is generally described in FIG. 3 and in Beigelman et al., International PCT publication No. WO 99/55857 and U.S. patent application Ser. No. 09/918,728. Zinzymes possess endonuclease activity to cleave RNA substrates having a cleavage triplet including but not limited to YG/Y, where Y is uridine or cytidine, and G is guanosine and /“/” represents the cleavage site. Zinzymes can be chemically modified to increase nuclease stability through substitutions as are generally shown in FIG. 3, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group within its own nucleic acid sequence for activity. In particular embodiments the enzymatic nucleic acid molecule can have an attached linker(s) or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 4 and is generally reviewed in Usman et al., U.S. Pat. No., 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection, see Santoro et al., supra and as generally described in Joyce et al., U.S. Pat. No. 5,807,718. Additional DNAzyme motifs can be selected by using techniques similar to those described in these references, and hence, are within the scope of the present invention. DNAzymes of the invention can comprise nucleotides modified at the nucleic acid base, sugar, or phosphate backbone. Non-limiting examples of sugar modifications that can be used in DNAzymes of the invention include 2′-O-alkyl modifications such as 2′-O-methyl or 2′-O-allyl, 2′-C-alkyl modifications such as 2′-C-allyl, 2′-deoxy-2′-amino, 2′-halo modifications such as 2′-fluoro, 2′-chloro, or 2′-bromo, isomeric modifications such as arabinofuranose or xylofuranose based nucleic acids, and other sugar modifications such as 4′-thio or 4′-carbocyclic nucleic acids. Non-limiting examples of nucleic acid based modifications that can be used in DNAzymes of the invention include modified purine heterocycles, G-clamp heterocycles, and various modified pyrimidine cycles. Non-limiting examples of backbone modifications that can be used in DNAzymes of the invention include phosphorothioate, phosphorodithioate, phosphoramidate, and methylphosphonate internucleotide linkages. DNAzymes of the invention can comprise naturally occurring nucleic acids, chimeras of chemically modified and naturally occurring nucleic acids, or completely modified nucleic acids.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “equivalent” RNA to HIV is meant to include those naturally occurring RNA molecules having homology (partial or complete) to HIV nucleic acid or encoding for proteins with similar function as HIV proteins in various organisms, including human, rodent, primate, rabbit, pig, protozoans, fungi, plants, and other microorganisms and parasites. The equivalent RNA sequence also includes in addition to the coding region, regions such as 5′-untranslated region, 3 ′-untranslated region, introns, intron-exon junction and the like.

By “homology” is meant the nucleotide sequence of two or more nucleic acid molecules is partially or completely identical.

By “component” of HIV is meant a peptide or protein expressed from an HIV gene, for example nef, vif, tat, or rev viral gene products.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “gene” it is meant a nucleic acid that encodes an RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “decoy ” is meant a nucleic acid molecule, for example RNA or DNA, or aptamer that is designed to preferentially bind to a predetermined ligand. Such binding can result in the inhibition or activation of a target molecule. The decoy RNA or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy RNA can be designed to bind to HIV and block the binding of HIV or a decoy RNA can be designed to bind to HIV and prevent interaction with the HIV protein.

The term “short interfering RNA” or “siRNA” as used herein refers to a double stranded nucleic acid molecule capable of RNA interference “RNAi”, see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. As used herein, siRNA molecules need not be limited to those molecules containing only RNA, but further encompasses chemically modified nucleotides and non-nucleotides.

Several varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

Nucleic acid molecules that modulate expression of HIV-specific RNAs represent a therapeutic approach to treat a variety of inflammatory diseases and conditions, including but not limited to rheumatoid arthritis, restenosis, asthma, Crohn's disease, diabetes, obesity, autoimmune disease, lupus, multiple sclerosis, transplant/graft rejection, gene therapy applications, ischemia/reperfusion injury (CNS and myocardial), glomerulonephritis, sepsis, allergic airway inflammation, inflammatory bowel disease, infection, and any other inflammatory disease or condition which respond to the modulation of HIV expression.

The enzymatic nucleic acid molecule that cleave the specified sites in HIV-specific RNAs also represent a therapeutic approach to treat acquired immunodeficiency syndrome (AIDS) and/or any other disease, condition, or syndrome which respond to the modulation of HIV expression.

In one embodiment of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 2; Beigelman et al., U.S. Ser. No. 09/301,511) and Zinzyme (FIG. 3) (Beigelman et al., U.S. Ser. No. 09/301,511), all included by reference herein including drawings, can also be used in the present invention. These specific motifs or configurations are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In one embodiment of the present invention, a nucleic acid molecule of the instant invention can be between about 10 and 100 nucleotides in length. Exemplary enzymatic nucleic acid molecules of the invention are shown in Tables III to VIII. For example, enzymatic nucleic acid molecules of the invention are preferably between about 15 and 50 nucleotides in length, more preferably between about 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between about 15 and 40 nucleotides in length, more preferably between about 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between about 15 and 75 nucleotides in length, more preferably between about 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between about 10 and 40 nucleotides in length, more preferably between about 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for the nucleic acid molecule are of length and conformation sufficient and suitable for the nucleic acid molecule to interact with its target and/or catalyze a reaction contemplated herein. The length of the nucleic acid molecules of the instant invention are not limiting within the general limits stated.

Preferably, a nucleic acid molecule that modulates, for example, down-regulates HIV expression comprises between 12 and 100 bases complementary to a RNA molecule of HIV. Even more preferably, a nucleic acid molecule that modulates HIV replication or expression comprises between 14 and 24 bases complementary to a RNA molecule of HIV.

The invention provides a method for producing a class of nucleic acid-based gene modulating agents which exhibit a high degree of specificity for the RNA of a desired target. For example, the enzymatic nucleic acid molecule is preferably targeted to a highly conserved sequence region of target RNAs encoding HIV (specifically HIV genes) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., enzymatic nucleic acid molecules, siRNA, antisense, and/or DNAzymes) can be expressed from DNA and/or RNA vectors that are delivered to specific cells.

As used herein “cell” is used in its usual biological sense, and does not refer to an entire multicellular organism. A cell can, for example, be in vitro, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

By “HIV proteins” is meant, a peptide or protein comprising a component of HIV or a peptide or protein encoded by a HIV gene.

By “highly conserved sequence region” is meant, a nucleotide sequence of one or more regions in a target gene that does not vary significantly from one generation to the other or from one biological system to the other.

Nucleic acid-based inhibitors of HIV expression are useful for the prevention and/or treatment of acquired immunodeficiency disease (AIDS) and related diseases and conditions, including but not limited to Kaposi's sarcoma, lymphoma, cervical cancer, squamous cell carcinoma, cardiac myopathy, rheumatic diseases, and opportunistic infection, for example Pneumocystis carinii, Cytomegalovirus, Herpes simplex, Mycobacteria, Cryptococcus, Toxoplasma, Progressive multifocal leucoencepalopathy (Papovavirus), Mycobacteria, Aspergillus, Cryptococcus, Candida, Cryptosporidium, Isospora belli, Microsporidia and any other disease or condition which respond to the modulation of HIV expression.

By “related” is meant that the reduction of HIV expression (specifically HIV gene) RNA levels and thus reduction in the level of the respective protein relieves, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers. In certain embodiments, the enzymatic nucleic acid inhibitors comprise sequences, which are complementary to the substrate sequences in Tables III to VIII. Examples of such enzymatic nucleic acid molecules also are shown in Tables III to VIII. Examples of such enzymatic nucleic acid molecules consist essentially of sequences defined in these tables.

In another embodiment, the invention features antisense nucleic acid molecules, siRNA nucleic acid molecules, and 2-5A chimeras including sequences complementary to the substrate sequences shown in Tables III to VIII. Such nucleic acid molecules can include sequences as shown for the binding arms of the enzymatic nucleic acid molecules in Tables III to VIII. Similarly, triplex molecules can be provided targeted to the corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present which do not interfere with such cleavage. Thus, a core region can, for example, include one or more loop, stem-loop structure, or linker which does not prevent enzymatic activity. Thus, the underlined regions in the sequences in Table III and IV can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO 159), or any other Stem II region known in the art, or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers can be present that do not interfere with the function of the nucleic acid molecule.

Sequence X can be a linker of ≧2 nucleotides in length, preferably 3, 4, 5, 6, 7, 8, 9, 10, 15, 20, 26, 30, where the nucleotides can preferably be internally base-paired to form a stem of preferably ≧2 base pairs. Alternatively or in addition, sequence X can be a non-nucleotide linker. In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, the non-nucleotide linker X is as defined herein. Non-nucleotides as can include abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound which can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave an RNA or DNA molecule.

In another aspect of the invention, enzymatic nucleic acid molecules, siRNA nucleic acid molecules or antisense molecules that interact with target RNA molecules and down-regulate HIV (specifically HIV gene) activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, adeno-associated virus, retrovirus, adenovirus, or alphavirus. Preferably, the recombinant vectors capable of expressing the enzymatic nucleic acid molecules or antisense are delivered as described above, and persist in target cells. Alternatively, viral vectors can be used that provide for transient expression of enzymatic nucleic acid molecules or antisense. Such vectors can be repeatedly administered as necessary. Once expressed, the enzymatic nucleic acid molecules or antisense bind to the target RNA and down-regulate its function or expression. Delivery of enzymatic nucleic acid molecule or antisense expressing vectors can be systemic, such as by intravenous or intramuscular administration, by administration to target cells ex-planted from the subject followed by reintroduction into the subject, or by any other means that would allow for introduction into the desired target cell. Antisense DNA and DNAzymes can be expressed via the use of a single stranded DNA intracellular expression vector.

By “vectors” is meant any nucleic acid- and/or viral-based technique used to deliver a desired nucleic acid.

By “subject” or “patient” is meant an organism, which is a donor or recipient of explanted cells or the cells of the organism. “Subject” or “Patient”″ also refers to an organism to which the nucleic acid molecules of the invention can be administered. Preferably, a subject or patient is a mammal or mammalian cells. More preferably, a subject or patient is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme, for example, with a nucleic acid molecule comprising chemical modifications. In some cases, the activity or stability of the nucleic acid molecule can be decreased (i.e., less than ten-fold), but the overall activity of the nucleic acid molecule is enhanced, in vivo.

The nucleic acid molecules of the instant invention, individually, or in combination or in conjunction with other drugs, can be used to treat diseases or conditions discussed above. For example, to treat a disease or condition associated with the levels of HIV, the subject can be treated, or other appropriate cells can be treated, as is evident to those skilled in the art, individually or in combination with one or more drugs under conditions suitable for the treatment.

In a further embodiment, the described molecules, such as antisense, siRNA, or enzymatic nucleic acids, can be used in combination with other known treatments to treat conditions or diseases discussed above. For example, the described molecules can be used in combination with one or more known therapeutic agents to treat acquired immunodeficiency disease (AIDS) and related diseases and conditions, including but not limited to Kaposi's sarcoma, lymphoma, cervical cancer, squamous cell carcinoma, cardiac myopathy, rheumatic diseases, and opportunistic infection, for example Pneumocystis carinii, Cytomegalovirus, Herpes simplex, Mycobacteria, Cryptococcus, Toxoplasma, Progressive multifocal leucoencepalopathy (Papovavirus), Mycobacteria, Aspergillus, Cryptococcus, Candida, Cryptosporidium, Isospora belli, Microsporidia and any other disease or condition which respond to the modulation of HIV expression.

In another embodiment, the invention features nucleic acid-based inhibitors (e.g., enzymatic nucleic acid molecules (eg; ribozymes), antisense nucleic acids, 2-5A antisense chimeras, triplex DNA, antisense nucleic acids containing RNA cleaving chemical groups) and methods for their use to down regulate or inhibit the expression of genes (e.g., HIV genes) capable of progression and/or maintenance of AIDS and/or other disease states which respond to the modulation of HIV expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”.

Other features and advantages of the invention will be apparent from the following description of the preferred embodiments thereof, and from the claims.

Mechanism of Action of Nucleic Acid Molecules of the Invention as is Known in the Art

Antisense: Antisense molecules can be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). Backbone modified DNA chemistry which have been thus far been shown to act as substrates for RNase H are phosphorothioates, phosphorodithioates, and borontrifluoridates. In addition, 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

RNA interference: RNA interference refers to the process of sequence specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence homologous to the siRNA. Cleavage of the target RNA takes place in the middle of the region complementary to the guide sequence of the siRNA duplex (Elbashir et al., 2001, Genes Dev., 15, 188).

Short interfering RNA mediated RNAi has been studied in a variety of systems. Fire et al., 1998, Nature, 391, 806, were the first to observe RNAi in C. Elegans. Wianny and Goetz, 1999, Nature Cell Biol., 2, 70, describes RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain requirements for siRNA length, structure, chemical composition, and sequence that are essential to mediate efficient RNAi activity. These studies have shown that 21 nucleotide siRNA duplexes are most active when containing two nucleotide 3′-overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309), however siRNA molecules lacking a 5′-phosphate are active when introduced exogenously, suggesting that 5′-phosphorylation of siRNA constructs may occur in vivo.

Enzymatic Nucleic Acid: Several varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention can modulate, e.g., down-regulate, HIV protein expression and can be used to treat disease or diagnose disease associated with the levels of HIV. Enzymatic nucleic acid sequences targeting HIV RNA and sequences that can be targeted with nucleic acid molecules of the invention to down-regulate HIV expression are shown in Tables III to VIII.

The enzymatic nature of an enzymatic nucleic acid molecule allows the concentration of enzymatic nucleic acid molecule necessary to affect a therapeutic treatment to be lower than a nucleic acid molecule lacking enzymatic activity. This reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a enzymatic nucleic acid molecule.

Nucleic acid molecules having an endonuclease enzymatic activity are able to repeatedly cleave other separate RNA molecules in a nucleotide base sequence-specific manner. With proper design and construction, such enzymatic nucleic acid molecules can be targeted to virtually any RNA transcript, and achieved efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

Enzymatic nucleic acid molecules of the invention that are allosterically regulated (“allozymes”) can be used to modulate, including down-regulate, HIV expression. These allosteric enzymatic nucleic acids or allozymes (see for example George et al., U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, for example, mutant HIV protein, wild-type HIV protein, mutant HIV RNA, wild-type HIV RNA, other proteins and/or RNAs involved in HIV activity, compounds, metals, polymers, molecules and/or drugs that are targeted to HIV expressing cells etc., which in turn modulates the activity of the enzymatic nucleic acid molecule. In response to interaction with a predetermined signaling agent, the allosteric enzymatic nucleic acid molecule's activity is activated or inhibited such that the expression of a particular target is selectively down-regulated. The target can comprise wild-type HIV, mutant HIV, a component of HIV, and/or a predetermined cellular component that modulates HIV activity. In a specific example, allosteric enzymatic nucleic acid molecules that are activated by interaction with a RNA encoding a mutant HIV protein are used as therapeutic agents in vivo. The presence of RNA encoding the mutant HIV protein activates the allosteric enzymatic nucleic acid molecule that subsequently cleaves the RNA encoding a mutant HIV protein resulting in the inhibition of mutant HIV protein expression. In this manner, cells that express the mutant form of the HIV protein are selectively targeted.

In another non-limiting example, an allozyme can be activated by a HIV protein, peptide, or mutant polypeptide that caused the allozyme to inhibit the expression of a HIV gene, by, for example, cleaving RNA encoded by a HIV gene. In this non-limiting example, the allozyme acts as a decoy to inhibit the function of HIV and also inhibit the expression of HIV once activated by the HIV protein.

Target Sites

Targets for useful enzymatic nucleic acid molecules and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, below are provided specific non-limiting examples of such methods, not limiting to those in the art. Enzymatic nucleic acid molecules to such targets are designed as described in the above applications and synthesized to be tested in vitro and in vivo, as also described. The sequences of human HIV RNAs were screened for optimal enzymatic nucleic acid target sites using a computer-folding algorithm. HIV-1 sequences were screened for conserved sequences across 111 HIV-1 genomic sequences shown in Table IX. Hammerhead, DNAzyme, Inozyme, Amberzyme, Zinzyme, or G-Cleaver enzymatic nucleic acid molecule binding/cleavage sites were identified. These sites are shown in Tables III to VIII (all sequences are 5′ to 3′ in the tables; underlined regions can be any sequence “X” or linker X, the actual sequence is not relevant here). The nucleotide base position is noted in the Tables as that site to be cleaved by the designated type of enzymatic nucleic acid molecule. Hhuman sequences can be screened and enzymatic nucleic acid molecule and/or antisense thereafter designed, as discussed in Stinchcomb et al., WO 95/23225. In addition, mouse targeted nucleic acid molecules can be useful to test efficacy of action of the enzymatic nucleic acid molecule, siRNA and/or antisense prior to testing in humans.

In addition, enzymatic nucleic acid, siRNA, and antisense nucleic acid molecule binding/cleavage sites were identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions, such as between, for example, the binding arms and the catalytic core of an enzymatic nucleic acid, are eliminated from consideration. Varying binding arm lengths can be chosen to optimize activity.

Antisense, hammerhead, DNAzyme, NCH, amberzyme, zinzyme or G-Cleaver enzymatic nucleic acid molecule, siRNA, and antisense nucleic acid binding/cleavage sites were identified and were designed to anneal to various sites in the RNA target. The enzymatic nucleic acid binding arms or siRNA and antisense nucleic acid sequences are complementary to the target site sequences described above. The nucleic acid molecules are chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic Acid Molecules

Synthesis of nucleic acids greater than 100 nucleotides in length can be difficult using automated methods, and the therapeutic cost of such molecules can be prohibitive. In this invention, small nucleic acid motifs (“small refers to nucleic acid motifs less than about 100 nucleotides in length, preferably less than about 80 nucleotides in length, and more preferably less than about 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides (e.g., antisense GeneBlocs) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the antisense oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for RNA and chemically modified RNA including certain enzymatic nucleic acid molecules and siRNA molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA•3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA•3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive nucleic acid molecules or binding attenuated control (BAC) oligonucleotides can be synthesized by substituting one or more nucleotides in the nucleic acid molecule to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Enzymatic nucleic acid molecules are purified by gel electrophoresis using known methods or are purified by high pressure liquid chromatography (HPLC; See Wincott et al., Supra, the totality of which is hereby incorporated herein by reference) and are re-suspended in water.

The sequences of the nucleic acid molecules, including enzymatic nucleic acid molecules and antisense, that are chemically synthesized, are shown in Table VIII. The sequences of the enzymatic nucleic acid and antisense constructs that are chemically synthesized, are complementary to the Target sequences shown in Table VIII. Those in the art will recognize that these sequences are representative only of many more such sequences where the enzymatic portion of the ribozyme (all but the binding arms) is altered to affect activity. The enzymatic nucleic acid and antisense construct sequences listed in Tables III to VIII can be formed of ribonucleotides or other nucleotides or non-nucleotides. Such enzymatic nucleic acid molecules with enzymatic activity are equivalent to the enzymatic nucleic acid molecules described specifically in the Tables.

Optimizing Activity of the Nucleic Acid Molecule of the Invention.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra, all of which are hereby incorporated by reference in their entirety). All of the above references describe various chemical modifications that can be made to the base, phosphate and/or sugar moieties of the nucleic acid molecules described herein. Modifications which enhance their efficacy in cells, and removal of bases from nucleic acid molecules to shorten oligonucleotide synthesis times and reduce chemical requirements are desired.

There are several examples of sugar, base and phosphate modifications that can be introduced into nucleic acid molecules with significant enhancement in their nuclease stability and efficacy. For example, oligonucleotides can be modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules are also known to increase efficacy (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). The publications describe general methods and strategies to determine the location of incorporation of sugar, base and/or phosphate modifications and the like into enzymatic nucleic acid molecules without inhibiting catalysis. Similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, excessive modifications can cause some toxicity. Therefore, when designing nucleic acid molecules, the amount of these internucleotide linkages should be minimized. The reduction in the concentration of these linkages can lower toxicity, resulting in increased efficacy and higher specificity of the therapeutic nucleic acid molecules.

Nucleic acid molecules having chemical modifications that maintain or enhance activity are provided. Such nucleic acid molecules are also generally more resistant to nucleases than unmodified nucleic acid molecules. Thus, the in vitro and/or in vivo activity should not be significantly lowered. Therapeutic nucleic acid molecules delivered exogenously are optimally stable within cells until translation of the target RNA has been inhibited long enough to reduce the levels of the undesirable protein. This period of time varies between hours to days, depending upon the disease state. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein)) have expanded the ability to modify nucleic acid molecules by introducing nucleotide modifications to enhance their nuclease stability as described above.

In one embodiment, nucleic acid molecules of the invention include one or more G-clamp nucleotides. A G-clamp nucleotide is a modified cytosine analog wherein modifications result in the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substation within an oligonucleotide can result in substantially enhanced helical thermal stability and mismatch discrimination when hybridized to complementary oligonucleotides. The inclusion of such nucleotides in nucleic acid molecules of the invention can enable both enhanced affinity and specificity to nucleic acid targets.

In another embodiment, the invention features conjugates and/or complexes of nucleic acid molecules targeting Ras genes such as K-Ras, H-Ras, and/or N-Ras. Compositions and conjugates are used to facilitate delivery of molecules into a biological system, such as cells. The conjugates provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel agents for the delivery of molecules, including but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example, enzymatic degradation or chemical degradation.

The term “biologically active molecule” as used herein, refers to compounds or molecules that are capable of eliciting or modifying a biological response in a system. Non-limiting examples of biologically active molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, dsRNA, allozymes, aptamers, decoys and analogs thereof. Biologically active molecules of the invention also include molecules capable of modulating the pharmacokinetics and/or pharmacodynamics of other biologically active molecules, for example lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus containing group and saturated or unsaturated alkyl group, optionally substituted with OH, COOH, oxo, amine, or substituted or unsubstituted aryl groups.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients or subjects with nucleic acid molecules can also include combinations of different types of nucleic acid molecules.

In the case that down-regulation of the target is desired, therapeutic nucleic acid molecules (e.g., DNAzymes) delivered exogenously are optimally stable within cells until translation of the target RNA has been inhibited long enough to reduce the levels of the targeted protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and others known in the art have expanded the ability to modify nucleic acid molecules by introducing nucleotide modifications to enhance their nuclease stability as described above.

In another embodiment, nucleic acid catalysts having chemical modifications that maintain or enhance enzymatic activity are provided. Such nucleic acids are also generally more resistant to nucleases than unmodified nucleic acid. Thus, the in vitro and/or in vivo the activity of the nucleic acid should not be significantly lowered. As exemplified herein, such enzymatic nucleic acids are useful for in vitro and/or in vivo techniques even if activity over all is reduced 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′-cap structure.

By “cap structure” is meant chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In another embodiment the 3′-cap includes, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

The term “alkyl” as used herein refers to a saturated aliphatic hydrocarbon, including straight-chain, branched-chain “isoalkyl”, and cyclic alkyl groups. The term “alkyl” also comprises alkoxy, alkyl-thio, alkyl-thio-alkyl, alkoxyalkyl, alkylamino, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Preferably, the alkyl group has 1 to 12 carbons. More preferably it is a lower alkyl of from about 1 to 7 carbons, more preferably about 1 to 4 carbons. The alkyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkenyl groups containing at least one carbon-carbon double bond, including straight-chain, branched-chain, and cyclic groups. Preferably, the alkenyl group has about 2 to 12 carbons. More preferably it is a lower alkenyl of from about 2 to 7 carbons, more preferably about 2 to 4 carbons. The alkenyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkynyl groups containing at least one carbon-carbon triple bond, including straight-chain, branched-chain, and cyclic groups. Preferably, the alkynyl group has about 2 to 12 carbons. More preferably it is a lower alkynyl of from about 2 to 7 carbons, more preferably about 2 to 4 carbons. The alkynyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Alkyl groups or moieties of the invention can also include aryl, alkylaryl, carbocyclic aryl, heterocyclic aryl, amide and ester groups. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from about 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

The term “alkoxyalkyl” as used herein refers to an alkyl-O-alkyl ether, for example methoxyethyl or ethoxymethyl.

The term “alkyl-thio-alkyl” as used herein refers to an alkyl-S-alkyl thioether, for example methylthiomethyl or methylthioethyl.

The term “amino” as used herein refers to a nitrogen containing group as is known in the art derived from ammonia by the replacement of one or more hydrogen radicals by organic radicals. For example, the terms “aminoacyl” and “aminoalkyl” refer to specific N-substituted organic radicals with acyl and alkyl substituent groups respectively.

The term “amination” as used herein refers to a process in which an amino group or substituted amine is introduced into an organic molecule.

The term “exocyclic amine protecting moiety” as used herein refers to a nucleobase amino protecting group compatible with oligonucleotide synthesis, for example an acyl or amide group.

The term “alkenyl” as used herein refers to a straight or branched hydrocarbon of a designed number of carbon atoms containing at least one carbon-carbon double bond. Examples of “alkenyl” include vinyl, allyl, and 2-methyl-3-heptene.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkenyl” as used herein refers to a C3-C8 cyclic hydrocarbon containing at least one carbon-carbon double bond. Examples of cycloalkenyl include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “cycloalkyl” as used herein refers to a C3-C8 cyclic hydrocarbon. Examples of cycloalkyl include cyclopropyl, cyclobutyl, cyclopentyl, cyclohexyl, cycloheptyl and cyclooctyl.

The term “cycloalkylalkyl,” as used herein, refers to a C3-C7 cycloalkyl group attached to the parent molecular moiety through an alkyl group, as defined above. Examples of cycloalkylalkyl groups include cyclopropylmethyl and cyclopentylethyl.

The terms “halogen” or “halo” as used herein refers to indicate fluorine, chlorine, bromine, and iodine.

The term “heterocycloalkyl,” as used herein refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “C1-C6 hydrocarbyl” as used herein refers to straight, branched, or cyclic alkyl groups having 1-6 carbon atoms, optionally containing one or more carbon-carbon double or triple bonds. Examples of hydrocarbyl groups include, for example, methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, vinyl, 2-pentene, cyclopropylmethyl, cyclopropyl, cyclohexylmethyl, cyclohexyl and propargyl. When reference is made herein to C1-C6 hydrocarbyl containing one or two double or triple bonds it is understood that at least two carbons are present in the alkyl for one double or triple bond, and at least four carbons for two double or triple bonds.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. For example, such modifications can enhance shelf-life, half-life in vitro, stability, and ease of introduction of such oligonucleotides to the target site, including e.g., enhancing penetration of cellular membranes and conferring the ability to recognize and bind to targeted cells.

Use of these molecules can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules). The treatment of subjects with nucleic acid molecules can also include combinations of different types of nucleic acid molecules. Therapies can be devised which include a mixture of enzymatic nucleic acid molecules (including different enzymatic nucleic acid molecule motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. Neuro Virol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a subject.

The negatively charged polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for exaple the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient or subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the invention, RNA molecules of the present invention are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the subject followed by reintroduction into the subject, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention is disclosed. The nucleic acid sequence encoding the nucleic acid molecule of the instant invention is operably linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, hammerhead, DNAzyme, NCH, Amberzyme, Zinzyme, or G-Cleaver ribozyme molecules and binding/cleavage sites within HIV RNA.

### Example 1

Identification of Potential Target Sites in Human HIV RNA

The sequence of human HIV genes were screened for accessible sites using a computer-folding algorithm. Regions of the RNA that do not form secondary folding structures and contained potential enzymatic nucleic acid molecule and/or antisense binding/cleavage sites were identified. The sequences of these binding/cleavage sites are shown in Tables III to VIII.

### Example 2

Selection of Enzymatic Nucleic Acid Cleavage Sites in Human HIV RNA

Enzymatic nucleic acid molecule target sites were chosen by analyzing sequences of Human HIV (Genbank accession No: NM_{—}005228) and prioritizing the sites on the basis of folding. Enzymatic nucleic acid molecules were designed that can bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core were eliminated from consideration. As noted below, varying binding arm lengths can be chosen to optimize activity. Generally, at least 5 bases on each arm are able to bind to, or otherwise interact with, the target RNA.

### Example 3

Chemical Synthesis and Purification of Ribozymes and Antisense for Efficient Cleavage and/or blocking of HIV Activity

Enzymatic nucleic acid molecules and antisense constructs are designed to anneal to various sites in the RNA message. The binding arms of the enzymatic nucleic acid molecules are complementary to the target site sequences described above, while the antisense constructs are fully complementary to the target site sequences described above. The enzymatic nucleic acid molecules and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Enzymatic nucleic acid molecules and antisense constructs are also synthesized from DNA templates using bacteriophage T7 RNA polymerase (Milligan and Uhlenbeck, 1989, Methods Enzymol. 180, 51). Enzymatic nucleic acid molecules and antisense constructs are purified by gel electrophoresis using general methods or are purified by high pressure liquid chromatography (HPLC; See Wincott et al., supra; the totality of which is hereby incorporated herein by reference) and are resuspended in water. The sequences of the chemically synthesized enzymatic nucleic acid molecules used in this study are shown below in Table VIII. The sequences of the chemically synthesized antisense constructs used in this study are complementary sequences to the Substrate sequences shown below as in Tables III to VIII.

### Example 4

Enzymatic Nucleic Acid Molecule Cleavage of HIV RNA Target in Vitro

Enzymatic nucleic acid molecules targeted to the human HIV RNA are designed and synthesized as described above. These enzymatic nucleic acid molecules can be tested for cleavage activity in vitro, for example, using the following procedure. The target sequences and the nucleotide location within the HIV RNA are given in Tables III to VIII.

Cleavage Reactions: Full-length or partially full-length, internally-labeled target RNA for enzymatic nucleic acid molecule cleavage assay is prepared by in vitro transcription in the presence of [a-³²P] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2× concentration of purified enzymatic nucleic acid molecule in enzymatic nucleic acid molecule cleavage buffer (50 mM Tris-HCl, pH 7.5 at 37° C., 10 mM MgCl₂) and the cleavage reaction was initiated by adding the 2× enzymatic nucleic acid molecule mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM enzymatic nucleic acid molecule, i.e., enzymatic nucleic acid molecule excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by enzymatic nucleic acid molecule cleavage are visualized on an autoradiograph of the gel. The percentage of cleavage is determined by Phosphor Imager® quantitation of bands representing the intact substrate and the cleavage products.

Indications

Particular degenerative and disease states that can be associated with HIV expression modulation include but are not limited to acquired immunodeficiency disease (AIDS) and related diseases and conditions, including but not limited to Kaposi's sarcoma, lymphoma, cervical cancer, squamous cell carcinoma, cardiac myopathy, rheumatic diseases, and opportunistic infection, for example Pneumocystis carinii, Cytomegalovirus, Herpes simplex, Mycobacteria, Cryptococcus, Toxoplasma, Progressive multifocal leucoencepalopathy (Papovavirus), Mycobacteria, Aspergillus, Cryptococcus, Candida, Cryptosporidium, Isospora belli, Microsporidia and any other diseases or conditions that are related to or will respond to the levels of HIV in a cell or tissue, alone or in combination with other therapies

The present body of knowledge in HIV research indicates the need for methods to assay HIV activity and for compounds that can regulate HIV expression for research, diagnostic, and therapeutic use.

The use of antiviral compounds, monoclonal antibodies, chemotherapy, radiation therapy, analgesics, and/or anti-inflammatory compounds, are all non-limiting examples of a methods that can be combined with or used in conjunction with the nucleic acid molecules (e.g. ribozymes and antisense molecules) of the instant invention. Examples of antiviral compounds that can be used in conjunction with the nucleic acid molecules of the invention include but are not limited to AZT (also known as zidovudine or ZDV), ddC (zalcitabine), ddI (dideoxyinosine), d4T (stavudine), and 3TC (lamivudine) Ribavirin, delvaridine (Rescriptor), nevirapine (Viramune), efravirenz (Sustiva), ritonavir (Norvir), saquinivir (Invirase), indinavir (Crixivan), amprenivir (Agenerase), nelfinavir (Viracept), and/or lopinavir (Kaletra). Common chemotherapies that can be combined with nucleic acid molecules of the instant invention include various combinations of cytotoxic drugs to kill cancer cells. These drugs include but are not limited to paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be similarly be readily combined with the nucleic acid molecules of the instant invention (e.g. ribozymes, siRNA and antisense molecules) are hence within the scope of the instant invention.

Diagnostic Uses

The nucleic acid molecules of this invention (e.g., enzymatic nucleic acid molecules) can be used as diagnostic tools to examine genetic drift and mutations within diseased cells or to detect the presence of HIV RNA in a cell. The close relationship between enzymatic nucleic acid molecule activity and the structure of the target RNA allows the detection of mutations in any region of the molecule which alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with HIV-related condition. Such RNA is detected by determining the presence of a cleavage product after treatment with an enzymatic nucleic acid molecule using standard methodology.

In a specific example, enzymatic nucleic acid molecules which cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., HIV) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is more fully described in George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

Additional Uses

Potential uses of sequence-specific enzymatic nucleic acid molecules of the instant invention can have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the claims that follow.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate cleavage
products with 3′-OH and 5′-guanosine.
Additional protein cofactors required in some cases to help folding and
maintenance of the active structure.
Over 300 known members of this class. Found as an intervening sequence in
Tetrahymena thermophila rRNA, fungal mitochondria, chloroplasts, phage T4, blue-
green algae, and others.
Major structural features largely established though phylogenetic comparisons,
mutagenesis, and biochemical studies [ⁱ,ⁱⁱ].
Complete kinetic framework established for one ribozyme [ⁱⁱⁱ,^{iv},^{v},^{vi}].
Studies of ribozyme folding and substrate docking underway [^{vii},^{viii},^{ix}].
Chemical modification investigation of important residues well established [^{x},^{xi}].
The small (4-6 nt) binding site may make this ribozyme too non-specific for
targeted RNA cleavage, however, the Tetrahymena group I intron has been used
to repair a “defective” β-galactosidase message by the ligation of new β-
galactosidase sequences onto the defective message [^{xii}].
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii}].
Reaction mechanism: possible attack by M²⁺-OH to generate cleavage products
with 3′-OH and 5′-phosphate.
RNAse P is found throughout the prokaryotes and eukaryotes. The RNA subunit
has been sequenced from bacteria, yeast, rodents, and primates.
Recruitment of endogenous RNAse P for therapeutic applications is possible
through hybridization of an External Guide Sequence (EGS) to the target RNA
[^{xiv},^{xv}]
Important phosphate and 2′OH contacts recently identified [^{xvi},^{xvii}]
Group II Introns
Size: >1000 nucleotides.
Trans cleavage of target RNAs recently demonstrated [^{xviii},^{xix}].
Sequence requirements not fully determined.
Reaction mechanism: 2′-OH of an internal adenosine generates cleavage products
with 3′-OH and a “lariat” RNA containing a 3′-5′ and a 2′-5′ branch point.
Only natural ribozyme with demonstrated participation in DNA cleavage [^{xx},^{xxi}] in
addition to RNA cleavage and ligation.
Major structural features largely established through phylogenetic comparisons
[^{xxii}].
Important 2′OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′to the scissile bond to generate cleavage
products with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate cleavage
products with 2′,3′-cyclic phosphate and 5′-OH ends.
14 known members of this class. Found in a number of plant pathogens
(virusoids) that use RNA as the infectious agent.
Essential structural features largely defined, including 2 crystal structures [^{xxvi},^{xxvii}]
Minimal ligation activity demonstrated (for engineering through in vitro selection)
[^{xxviii}]
Complete kinetic framework established for two or more ribozymes [^{xxix}].
Chemical modification investigation of important residues well established [^{xxx}].
Hairpin Ribozyme
Size: ˜50 nucleotides.
Requires the target sequence GUC immediately 3′of the cleavage site.
Binds 4-6 nucleotides at the 5′-side of the cleavage site and a variable number to
the 3′-side of the cleavage site.
Reaction mechanism: attack by 2′-OH 5′ to the scissile bond to generate cleavage
products with 2′,3′-cyclic phosphate and 5′-OH ends.
3 known members of this class. Found in three plant pathogen (satellite RNAs of
the tobacco ringspot virus, arabis mosaic virus and chicory yellow mottle virus)
which uses RNA as the infectious agent.
Essential structural features largely defined [^{xxxi},^{xxxii},^{xxxiii},^{xxxiv}]
Ligation activity (in addition to cleavage activity) makes ribozyme amenable to
engineering through in vitro selection [^{xxxv}]
Complete kinetic framework established for one ribozyme [^{xxxvi}].
Chemical modification investigation of important residues begun [^{xxxvii},^{xxxviii}].
Hepatitis Delta Virus (HDV) Ribozyme
Size: ˜60 nucleotides.
Trans cleavage of target RNAs demonstrated [^{xxxix}].
Binding sites and structural requirements not fully determined, although no
sequences 5′ of cleavage site are required. Folded ribozyme contains a pseudoknot
structure [^{xl}].
Reaction mechanism: attack by 2′-OH 5′ to the scissile bond to generate cleavage
products with 2′,3′-cyclic phosphate and 5′-OH ends.
Only 2 known members of this class. Found in human HDV.
Circular form of HDV is active and shows increased nuclease stability [^{xli}]
ⁱMichel, Francois; Westhof, Eric. Slippery substrates. Nat. Struct. Biol. (1994), 1(1), 5-7.
ⁱⁱLisacek, Frederique; Diaz, Yolande; Michel, Francois. Automatic identification of group I intron cores in genomic DNA sequences. J. Mol. Biol. (1994), 235(4), 1206-17.
ⁱⁱⁱHerschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.
^{iv}Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
^{v}Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
^{vi}Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.
^{vii}Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
^{viii}Banerjee, Aloke Raj; Turner, Douglas H.. The time dependence of chemical modification reveals slow steps in the folding of a group I ribozyme. Biochemistry (1995), 34(19), 6504-12.
^{ix}Zarrinkar, Patrick P.; Williamson, James R.. The P9.1-P9.2 peripheral extension helps guide folding of the Tetrahymena ribozyme. Nucleic Acids Res. (1996), 24(5), 854-8.
^{x}Strobel, Scott A.; Cech, Thomas R.. Minor groove recognition of the conserved G.cntdot.U pair at the Tetrahymena ribozyme reaction site. Science (Washington, D. C.) (1995), 267(5198), 675-9.
^{xi}Strobel, Scott A.; Cech, Thomas R.. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
^{xii}Sullenger, Bruce A.; Cech, Thomas R.. Ribozyme-mediated repair of defective mRNA by targeted trans-splicing. Nature (London) (1994), 371(6498), 619-22.
^{xiii}Robertson, H. D.; Altman, S.; Smith, J.D. J. Biol. Chem., 247, 5243-5251 (1972).
^{xiv}Forster, Anthony C.; Altman, Sidney. External guide sequences for an RNA enzyme. Science (Washington, D. C., 1883-) (1990), 249(4970), 783-6.
^{xv}Yuan, Y.; Hwang, E. S.; Altman, S. Targeted cleavage of mRNA by human RNase P. Proc. Natl. Acad. Sci. USA (1992) 89, 8006-10.
^{xvi}Harris, Michael E.; Pace, Norman R.. Identification of phosphates involved in catalysis by the ribozyme RNase P RNA. RNA (1995), 1(2), 210-18.
^{xvii}Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U. S. A. (1995), 92(26), 12510-14.
^{xviii}Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
^{xix}Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
^{xx}Zimmerly, Steven; Guo, Huatao; Eskes, Robert; Yang, Jian; Perlman, Philip S.; Lambowitz, Alan M.. A group II intron ENA is a catalytic component of a DNA endonuclease involved in intron mobility. Cell (Cambridge, Mass.) (1995), 83(4), 529-38.
^{xxi}Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′- hydroxyl groups. Chem. Biol. (1995), 2(11), 761-70.
^{xxii}Michel, Francois; Ferat, Jean Luc. Structure and activities of group II introns. Annu. Rev. Biochem. (1995), 64,435-61.
^{xxiii}Abramovitz, Dana L.; Friedman, Richard A.; Pyle, Anna Marie. Catalytic role of 2′-hydroxyl groups within a group II intron active site. Science (Washington, D. C.) (1996), 271(5254), 1410-13.
^{xxiv}Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
^{xxv}Guo, Hans C. T.; Collins, Richard A.. Efficient trans-cleavage of a stem-loop RNA substrate by a ribozyme derived from Neurospora VS RNA. EMBO J. (1995), 14(2), 368-76.
^{xxvi}Scott, W.G., Finch, J.T., Aaron, K. The crystal structure of an all RNA hammerhead ribozyme:Aproposed mechanism for RNA catalytic cleavage. Cell, (1995), 81, 991-1002.
^{xxvii}McKay, Structure and function of the hammerhead ribozyme: an unfinished story. RNA, (1996), 2, 395-403.
^{xxviii}Long, D., Uhlenbeck, O., Hertel, K. Ligation with hammerhead ribozymes. U.S. Pat. No. 5,633,133.
^{xxix}Hertel, K.J., Herschlag, D., Uhlenbeck, O. A kinetic and thermodynamic framework for the hammerhead ribozyme reaction. Biochemistry, (1994) 33, 3374-3385. Beigelman, L., et al., Chemical modifications of hammerhead ribozymes. J. Biol. Chem., (1995) 270, 25702-25708.
^{xxx}Beigelman, L., et al., Chemical modifications of hammerhead ribozymes. J. Biol. Chem., (1995) 270, 25702-25708.
^{xxxi}Hampel, Arnold; Tritz, Richard; Hicks, Margaret; Cruz, Phillip. ‘Hairpin’catalytic RNA model: evidence for helixes and sequence requirement for substrate RNA. Nucleic Acids Res. (1990), 18(2), 299-304.
^{xxxii}Chowrira, Bharat M.; Berzal-Herranz, Alfredo; Burke, John M.. Novel guanosine requirement for catalysis by the hairpin ribozyme. Nature (London) (1991), 354(6351), 320-2.
^{xxxiii}Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J. (1993), 12(6), 2567-73.
^{xxxiv}Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.
^{xxxv}Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.
^{xxxvi}Hegg, Lisa A.; Fedor, Martha J.. Kinetics and Thermodynamics of Intermolecular Catalysis by Hairpin Ribozymes. Biochemistry (1995), 34(48), 15813-28.
^{xxxvii}Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34(12), 4068-76.
^{xxxviii}Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.
^{xxxix}Perrotta, Anne T.; Been, Michael D.. Cleavage of oligoribonucleotides by a ribozyme derived from the hepatitis .delta. virus RNA sequence. Biochemistry (1992), 31(1), 16-21.
^{xl}Perrotta, Anne T.; Been, Michael D.. A pseudoknot-like structure required for efficient self-cleavage of hepatitis delta virus RNA. Nature (London) (1991), 350(6317), 434-6.
^{xli}Puttaraju, M.; Perrotta, Anne T.; Been, Michael D.. A circular trans-acting hepatitis delta virus ribozyme. Nucleic Acids Res. (1993), 21(18), 4253-8.

TABLE II
A. 2.5 μmol Synthesis Cycle ABI 394 Instrument

| Reagent | Equivalents | Amount | | Wait Time* DNA | | Wait Time* 2′-O-methyl | | Wait Time*RNA | |
|---|---|---|---|---|---|---|---|---|---|
| Phosphoramidites | 6.5 | 163 | μL | 45 | sec | 2.5 | min | 7.5 | min |
| S-Ethyl Tetrazole | 23.8 | 238 | μL | 45 | sec | 2.5 | min | 7.5 | min |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | mL | NA | NA | NA |

B. 0.2 μmol Synthesis Cycle ABI 394 Instrument

| Reagent | Equivalents | Amount | | Wait Time* DNA | | Wait Time* 2′-O-methyl | | Wait Time*RNA | |
|---|---|---|---|---|---|---|---|---|---|
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

C. 0.2 μmol Synthesis Cycle 96 well Instrument

| | Equivalents:DNA/ | Amount: DNA/2′-O- | | | | Wait Time* 2′-O- | | Wait Time* | |
|---|---|---|---|---|---|---|---|---|---|
| Reagent | 2′-O-methyl/Ribo | methyl/Ribo | | Wait Time* DNA | | methyl | | Ribo | |
| Phosphoramidites | 22/33/66 | 40/60/120 | μL | 60 | sec | 180 | sec | 360 | sec |
| S-Ethyl Tetrazole | 70/105/210 | 40/60/120 | μL | 60 | sec | 180 | min | 360 | sec |
| Acetic Anhydride | 265/265/265 | 50/50/50 | μL | 10 | sec | 10 | sec | 10 | sec |
| N-Methyl | 502/502/502 | 50/50/50 | μL | 10 | sec | 10 | sec | 10 | sec |
| Imidazole | | | | | | | | | |
| TCA | 238/475/475 | 250/500/500 | μL | 15 | sec | 15 | sec | 15 | sec |
| Iodine | 6.8/6.8/6.8 | 80/80/80 | μL | 30 | sec | 30 | sec | 30 | sec |
| Beaucage | 34/51/51 | 80/120/120 | | 100 | sec | 200 | sec | 200 | sec |

| | | | | | | |
|---|---|---|---|---|---|---|
| Acetonitrile | NA | 1150/1150/1150 | μL | NA | NA | NA |

*Wait time does not include contact time during delivery.

TABLE III
Human HIV Hammerhead Ribozyme
and Substrate Sequence

| | Seq | | Seq |
|---|---|---|---|
| Substrate | ID | Hammerhead | ID |
| AUAAAGCU U | 1 | CUCAAGGC CUGAUGAGGCCGUUAGGCCGAA | 77 |
| GCCUUGAG | | AGCUUUAU | |
| AGGCUAAU U | 2 | CCCUAAAA CUGAUGAGGCCGUUAGGCCGAA | 78 |
| UUUUAGGG | | AUUAGCCU | |
| GGCUAAUU U | 3 | UCCCUAAA CUGAUGAGGCCGUUAGGCCGAA | 79 |
| UUUAGGGA | | AAUUAGCC | |
| GCCUCAAU A | 4 | GCAAGCUU CUGAUGAGGCCGUUAGGCCGAA | 80 |
| AAGCUUGC | | AUUGAGGC | |
| UUUCGGGU U | 5 | CUGUAAUA CUGAUGAGGCCGUUAGGCCGAA | 81 |
| UAUUACAG | | ACCCGAAA | |
| GCAGGACU C | 6 | AGCAAGCC CUGAUGAGGCCGUUAGGCCGAA | 82 |
| GGCUUGCU | | AGUCCUGC | |

Input Sequence = HIV1.
Cut Site = UH/.
Arm Length = 8.
Core Sequence = CUGAUGAG GCCGUUAGGC CGAA
HIV1 Consensus

Underlined region can be any X sequence or linker, as described herein.

TABLE IV
Human HIV Inozyme and Substrate Sequence

| | Seq | | Seq |
|---|---|---|---|
| Substrate | ID | Inozyme | ID |
| UGGAAAAC A | 7 | CUGCCAUC CUGAUGAGGCCGUUAGGCCGAA | 83 |
| GAUGGCAG | | IUUUUCCA | |
| AAUAAAGC U | 8 | UCAAGGCA CUGAUGAGGCCGUUAGGCCGAA | 84 |
| UGCCUUGA | | ICUUUAUU | |
| UCUCUAGC A | 9 | GGCGCCAC CUGAUGAGGCCGUUAGGCCGAA | 85 |
| GUGGCGCC | | ICUAGAGA | |
| GGAGCCAC C | 10 | UCUUGUGG CUGAUGAGGCCGUUAGGCCGAA | 86 |
| CCACAAGA | | IUGGCUCC | |
| AGUGGCGC C | 11 | CCUGUUCG CUGAUGAGGCCGUUAGGCCGAA | 87 |
| CGAACAGG | | ICGCCACU | |
| GUGGCGCC C | 12 | CCCUGUUC CUGAUGAGGCCGUUAGGCCGAA | 88 |
| GAACAGGG | | IGCGCCAC | |
| CUCGACGC A | 13 | CCGAGUCC CUGAUGAGGCCGUUAGGCCGAA | 89 |
| GGACUCGG | | ICGUCGAG | |
| CGCAGGAC U | 14 | GCAAGCCG CUGAUGAGGCCGUUAGGCCGAA | 90 |
| CGGCUUGC | | IUCCUGCG | |

Input Sequence = HIV1.
Cut Site = CH/.
Arm Length = 8.
Core Sequence = CUGAUGAG GCCGUUAGGC CGAA
HIV1 Consensus

Underlined region can be any X sequence or linker, as described herein. “I” stands for Inosine.

TABLE V
Human HIV Zinzyme and Substrate Sequence

| | Seq | | Seq |
|---|---|---|---|
| Substrate | ID | Zinzyme | ID |
| UCAAUAAA G | 15 | AAGGCAAG GCCGAAAGGCGAGUGAGGUCU | 91 |
| CUUGCCUU | | UUUAUUGA | |
| AGGACUCG G | 16 | UCAGCAAG GCCGAAAGGCGAGUGAGGUCU | 92 |
| CUUGCUGA | | CGAGUCCU | |
| GCAGUGGC G | 17 | UGUUCGGG GCCGAAAGGCGAGUGAGGUCU | 93 |
| CCCGAACA | | GCCACUGC | |
| CUCUAGCA G | 18 | GGGCGCCA GCCGAAAGGCGAGUGAGGUCU | 94 |
| UGGCGCCC | | UGCUAGAG | |
| UAGCAGUG G | 19 | UUCGGGCG GCCGAAAGGCGAGUGAGGUCU | 95 |
| CGCCCGAA | | CACUGCUA | |
| AGAGAUGG G | 20 | CUCUCGCA GCCGAAAGGCGAGUGAGGUCU | 96 |
| UGCGAGAG | | CCAUCUCU | |
| AGAUGGGU G | 21 | CGCUCUCG GCCGAAAGGCGAGUGAGGUCU | 97 |
| CGAGAGCG | | ACCCAUCU | |
| CUCUCGAC G | 22 | GAGUCCUG GCCGAAAGGCGAGUGAGGUCU | 98 |
| CAGGACUC | | GUCGAGAG | |

Input Sequence = HIV1.
Cut Site = G/Y
Arm Length = 8.
Core Sequence = GCcgaaagGCGaGuCaaGGuCu
HIV1 Consensus

TABLE VI
Human HIV DNAzyme and Substrate Sequence

| | Seq | | Seq |
|---|---|---|---|
| Substrate | ID | DNAzyme | ID |
| UCAAUAAA G | 15 | AAGGCAAG GGCTAGCTACAACGA | 99 |
| CUUGCCUU | | TTTATTGA | |
| AGGACUCG G | 16 | TCAGCAAG GGCTAGCTACAACGA | 100 |
| CUUGCUGA | | CGAGTCCT | |
| GCAGUGGC G | 17 | TGTTCGGG GGCTAGCTACAACGA | 101 |
| CCCGAACA | | GCCACTGC | |
| CUCUAGCA G | 18 | GGGCGCCA GGCTAGCTACAACGA | 102 |
| UGGCGCCC | | TGCTAGAG | |
| UAGCAGUG G | 19 | TTCGGGCG GGCTAGCTACAACGA | 103 |
| CGCCCGAA | | CACTGCTA | |
| AGAGAUGG G | 20 | CTCTCGCA GGCTAGCTACAACGA | 104 |
| UGCGAGAG | | CCATCTCT | |
| AGAUGGGU G | 21 | CGCTCTCG GGCTAGCTACAACGA | 105 |
| CGAGAGCG | | ACCCATCT | |
| CUCUCGAC G | 22 | GAGTCCTG GGCTAGCTACAACGA | 106 |
| CAGGACUC | | GTCGAGAG | |
| UAUGGAAA A | 23 | GCCATCTG GGCTAGCTACAACGA | 107 |
| CAGAUGGC | | TTTCCATA | |
| GAAAACAG A | 24 | ACCTGCCA GGCTAGCTACAACGA | 108 |
| UGGCAGGU | | CTGTTTTC | |
| AAGCCUCA A | 25 | AAGCTTTA GGCTAGCTACAACGA | 109 |
| UAAAGCUU | | TGAGGCTT | |
| GGAGAGAG A | 26 | CGCACCCA GGCTAGCTACAACGA | 110 |
| UGGGUGCG | | CTCTCTCC | |
| GACGCAGG A | 27 | AAGCCGAG GGCTAGCTACAACGA | 111 |
| CUCGGCUU | | CCTGCGTC | |

Input Sequence = HIV1.
Cut Site = R/Y
Arm Length = 8.
Core Sequence = GGCTAGCTACAACGA
HIV1 Consensus

TABLE VII
Human HIV Amberzyme and Substrate Sequence

| Substrate | Seq ID | Amberzyme | Seq ID |
|---|---|---|---|
| UCAAUAAA G CUUGCCUU | 15 | AAGGCAAG GGAGGAAACUCC CU UCAAGGACAUCGUCCGGG UUUAUUGA | 112 |
| AGGACUCG G CUUGCUGA | 16 | UCAGCAAG GGAGGAAACUCC CU UCAAGGACAUCGUCCGGG CGAGUCCU | 113 |
| GCAGUGGC G CCCGAACA | 17 | UGUUCGGG GGAGGAAACUCC CU UCAAGGACAUCGUCCGGG GCCACUGC | 114 |
| CUCUAGCA G UGGCGCCC | 18 | GGGCGCCA GGAGGAAACUCC CU UCAAGGACAUCGUCCGGG UGCUAGAG | 115 |
| UAGCAGUG G CGCCCGAA | 19 | UUCGGGCG GGAGGAAACUCC CU UCAAGGACAUCGUCCGGG CACUGCUA | 116 |
| AGAGAUGG G UGCGAGAG | 20 | CUCUCGCA GGAGGAAACUCC CU UCAAGGACAUCGUCCGGG CCAUCUCU | 117 |
| AGAUGGGU G CGAGAGCG | 21 | CGCUCUCG GGAGGAAACUCC CU UCAAGGACAUCGUCCGGG ACCCAUCU | 118 |
| CUCUCGAC G CAGGACUC | 22 | GAGUCCUG GGAGGAAACUCC CU UCAAGGACAUCGUCCGGG GUCGAGAG | 119 |
| GGAAAACA G AUGGCAGG | 28 | CCUGCCAU GGAGGAAACUCC CU UCAAGGACAUCGUCCGGG UGUUUUCC | 120 |
| AUGGGUGC G AGAGCGUC | 29 | GACGCUCU GGAGGAAACUCC CU UCAAGGACAUCGUCCGGG GCACCCAU | 121 |
| AAAAGGGG G GAUUGGGG | 30 | CCCCAAUC GGAGGAAACUCC CU UCAAGGACAUCGUCCGGG CCCCUUUU | 122 |
| AGAAAAGG G GGGAUUGG | 31 | CCAAUCCC GGAGGAAACUCC CU UCAAGGACAUCGUCCGGG CCUUUUCU | 123 |
| GAAAAGGG G GGAUUGGG | 32 | CCCAAUCC GGAGGAAACUCC CU UCAAGGACAUCGUCCGGG CCCUUUUC | 124 |
| GGCUAGAA G GAGAGAGA | 33 | UCUCUCUC GGAGGAAACUCC CU UCAAGGACAUCGUCCGGG UUCUAGCC | 125 |
| UUUUAAAA G AAAAGGGG | 34 | CCCCUUUU GGAGGAAACUCC CU UCAAGGACAUCGUCCGGG UUUUAAAA | 126 |
| UAUGGCAG G AAGAAGCG | 35 | CGCUUCUU GGAGGAAACUCC CU UCAAGGACAUCGUCCGGG CUGCCAUA | 127 |
| UGGCGCCC G AACAGGGA | 36 | UCCCUGUU GGAGGAAACUCC CU UCAAGGACAUCGUCCGGG GGGCGCCA | 128 |
| GAGAGAUG G GUGCGAGA | 37 | UCUCGCAC GGAGGAAACUCC CU UCAAGGACAUCGUCCGGG CAUCUCUC | 129 |
| CGACGCAG G ACUCGGCU | 38 | AGCCGAGU GGAGGAAACUCC CU UCAAGGACAUCGUCCGGG CUGCGUCG | 130 |
| UGACUAGC G GAGGCUAG | 39 | CUAGCCUC GGAGGAAACUCC CU UCAAGGACAUCGUCCGGG GCUAGUCA | 131 |
| UAGAAGGA G AGAGAUGG | 40 | CCAUCUCU GGAGGAAACUCC CU UCAAGGACAUCGUCCGGG UCCUUCUA | 132 |
| AGGAGAGA G AUGGGUGC | 41 | GCACCCAU GGAGGAAACUCC CU UCAAGGACAUCGUCCGGG UCUCUCCU | 133 |
| GAAGGAGA G AGAUGGGU | 42 | ACCCAUCU GGAGGAAACUCC CU UCAAGGACAUCGUCCGGG UCUCCUUC | 134 |
| UCGACGCA G GACUCGGC | 43 | GCCGAGUC GGAGGAAACUCC CU UCAAGGACAUCGUCCGGG UGCGUCGA | 135 |
| CUAGCAGU G GCGCCCGA | 44 | UCGGGCGC GGAGGAAACUCC CU UCAAGGACAUCGUCCGGG ACUGCUAG | 136 |
| GACUAGCG G AGGCUAGA | 45 | UCUAGCCU GGAGGAAACUCC CU UCAAGGACAUCGUCCGGG CGCUAGUC | 137 |
| GCUAGAAG G AGAGAGAU | 46 | AUCUCUCU GGAGGAAACUCC CU UCAAGGACAUCGUCCGGG CUUCUAGC | 138 |
| AAAGGGGG G AUUGGGGG | 47 | CCCCCAAU GGAGGAAACUCC CU UCAAGGACAUCGUCCGGG CCCCCUUU | 139 |

Input Sequence = HIV1.
Cut Site = G/.
Arm Length = 8.
Core Sequence = GGAGGAAACUCC CU UCAAGGACAUCGUCCGGG
HIV1 Consensus

TABLE VIII
Human HIV Enzymatic Nucleic Acid and Target molecules

| Target | Seq ID | RPI# | Enzymatic Nucleic Acid | Seq ID |
|---|---|---|---|---|
| GAGAUGG G UGCGAGA | 140 | 25003 | ucucgca GGCTAGCTACAACGA ccaucuc B | 149 |
| AUGGAAA A CAGAUGG | 141 | 25004 | ccaucug GGCTAGCTACAACGA uuuccau B | 150 |
| AAAACAG A UGGCAGG | 142 | 25005 | ccugcca GGCTAGCTACAACGA cuguuuu B | 151 |
| AGCCUCA A UAAAGCU | 143 | 25006 | agcuuua GGCTAGCTACAACGA ugaggcu B | 152 |
| GAGAGAG A UGGGUGC | 144 | 25007 | gcaccca GGCTAGCTACAACGA cucucuc B | 153 |
| CAAUAAA G CUUGCCU | 145 | 25008 | aggcaag gccgaaaggCgagugaGGuCu uuuauug B | 154 |
| GGACUCG G CUUGCUG | 146 | 25009 | cagcaag gccgaaaggCgagugaGGuCu cgagucc B | 155 |
| GAGAUGG G UGCGAGA | 140 | 25010 | ucucgca gccgaaaggCgagugaGGuCu ccaucuc B | 156 |
| GAUGGGU G CGAGAGC | 147 | 25011 | gcucucg gccgaaaggCgagugaGGuCu acocauc B | 157 |
| UCUCGAC G CAGGACU | 148 | 25012 | aguccug gccgaaaggCgagugaGGuCu gucgaga B | 158 |

G = Guanosine
A, G, C, T (italic) = deoxy
lower case = 2′-O-methyl
s = phosphorothioate 3′-internucleotide linkage
C = 2′-deoxy-2′-Amino cytidine
B = inverted deoxyabasic derivative

TABLE IX
Human HIV-1 Sequences

| Genbank | | | |
|---|---|---|---|
| Acc# | Seq Name(s) | Subtype | Organism |
| A04321 | IIIB LAI | B | HIV-1 |
| AF110962 | 96BW0402 | C | HIV-1 |
| AF110963 | 96BW0407 | C | HIV-1 |
| AF110968 | 96BW0504 | C | HIV-1 |
| AF110965 | 96BW0409 | C | HIV-1 |
| AF110966 | 96BW0410 | C | HIV-1 |
| AF110964 | 96BW0408 | C | HIV-1 |
| AF110975 | 96BW15C05 | C | HIV-1 |
| AF110974 | 96BW15C02 | C | HIV-1 |
| AF110973 | 96BW15B03 | C | HIV-1 |
| AF107771 | UGSE8131 | A | HIV-1 |
| U69585 | WCIPR854 | B | HIV-1 |
| U69588 | WCIPR855 | B | HIV-1 |
| U69589 | WCIPR9011 | B | HIV-1 |
| U69591 | WCIPR9018 | B | HIV-1 |
| U69592 | WCIPR9031 | B | HIV-1 |
| U69593 | WCIPR9032 | B | HIV-1 |
| U69586 | WCIPR8546 | B | HIV-1 |
| AF003888 | NL43WC001 | B | HIV-1 |
| X01762 | REHTLV3 LAI IIIB | B | HIV-1 |
| AF075719 | MNTQ MNcloneTQ | B | HIV-1 |
| AJ239083 | 97CAMP645MO | MO | HIV-1 |
| D86069 | PM213 | B | HIV-1 |
| K02083 | PV22 | B | HIV-1 |
| M93259 | YU10 | B | HIV-1 |
| Z11530 | F12CG | B | HIV-1 |
| AB032740 | TH022 95TNIH022 | CRF01_AE | HIV-1 |
| AF107770 | SE7812 | CRF02_AG | HIV-1 |
| AF070521 | NL43E9 | B | HIV-1 |
| AF033819 | HXB2-copy LAI | B | HIV-1 |
| AF003887 | WC001 | B | HIV-1 |
| AF069140 | DH123 | B | HIV-1 |
| AF110967 | 96BW0502 | C | HIV-1 |
| K03455 | HXB2 HXB2CG | B | HIV-1 |
| M96155 | P896 89.6 | B | HIV-1 |
| X04415 | MAL MALCG | ADK | HIV-1 |
| AF133821 | MB2059 | D | HIV-1 |
| D86068 | MCK1 | B | HIV-1 |
| U69587 | WCIPR8552 | B | HIV-1 |
| U69590 | WCIPR9012 | B | HIV-1 |
| AB032741 | 95TNIH047 TH047 | CRF01_AE | HIV-1 |
| AB023804 | 93IN101 | C | HIV-1 |
| AF193275 | 97BL006 | A | HIV-1 |
| AF197340 | 90CF11697 | CRF01_AE | HIV-1 |
| AF224507 | WK | B | HIV-1 |
| AJ271445 | GB8 GB8-46R | B | HIV-1 |
| AF197338 | 93TH057 | CRF01_AE | HIV-1 |
| AF197339 | 93TH065 | CRF01_AE | HIV-1 |
| AF197341 | 90CF4071 | CRF01_AE | HIV-1 |
| U69584 | 85WCIPR54 | B | HIV-1 |
| L31963 | TH475A LAI | B | HIV-1 |
| U46016 | ETH2220 C2220 | C | HIV-1 |
| U21135 | WEAU160 GHOSH | B | HIV-1 |
| AF042106 | MBCC18R01 | B | HIV-1 |
| K03454 | ELI | D | HIV-1 |
| U51188 | 90CF402 90CR402 | CRF01_AE | HIV-1 |
| U51189 | 93TH253 | CRF01_AE | HIV-1 |
| U34603 | H0320-2A12 | B | HIV-1 |
| M38429 | JRCSF JR-CSF | B | HIV-1 |
| M17451 | RF HAT3 | B | HIV-1 |
| L02317 | BC BCSG3 | B | HIV-1 |
| M93258 | YU2 YU2X | B | HIV-1 |
| M22639 | Z2Z6 Z2 CDC-Z34 | D | HIV-1 |
| AF004394 | AD8, AD87 ADA | B | HIV-1 |
| AF049337 | 94CY032-3 | CRF04_cpx | HIV-1 |
| U34604 | 3202A21 | B | HIV-1 |
| L20587 | ANT70 | O | HIV-1 |
| D10112 | CAM1 | B | HIV-1 |
| U54771 | CM240 | CRF01_AE | HIV-1 |
| U43096 | D31 | B | HIV-1 |
| U37270 | C18MBC | B | HIV-1 |
| U43141 | HAN | B | HIV-1 |
| U23487 | MANC | B | HIV-1 |
| M17449 | MNCG MN | B | HIV-1 |
| L20571 | MVP5180 | O | HIV-1 |
| M27323 | NDK | D | HIV-1 |
| M38431 | NY5CG | B | HIV-1 |
| M26727 | OYI, 397 | B | HIV-1 |
| K02007 | SF2 LAV2 ARV2 | B | HIV-1 |
| M62320 | U455 U455A | A | HIV-1 |
| U26546 | WR27 | B | HIV-1 |
| AF004885 | Q23 | A | HIV-1 |
| AF042100 | MBC200 | B | HIV-1 |
| AF042101 | MBC925 | B | HIV-1 |
| AJ006287 | 89SP061 89ES061 | B | HIV-1 |
| AF067154 | 93IN999 301999 | C | HIV-1 |
| AF067155 | 95IN21068 21068 | C | HIV-1 |
| AJ006022 | YBF30 | N | HIV-1 |
| AF061642 | SE6165 G6165 | G | HIV-1 |
| AF119820 | 97PVCH GR11 | CRF04_cpx | HIV-1 |
| AF119819 | 97PVMY GR84 | CRF04_cpx | HIV-1 |
| K02013 | LAI BRU | B | HIV-1 |
| L39106 | IBNG | CRF02_AG | HIV-1 |
| U12055 | LW123 | B | HIV-1 |
| M19921 | NL43 pNL43 | B | HIV-1 |
| AF061640 | HH8793-1.1 | G | HIV-1 |
| AF061641 | HH8793-12.1 | G | HIV-1 |
| AF063223 | DJ263 | CRF02_AG | HIV-1 |
| AF049495 | NC7 | B | HIV-1 |
| AF049494 | 499JC16 | B | HIV-1 |
| AF086817 | TWCYS LM49 | B | HIV-1 |
| AF064699 | BFP90 | CRF06_cpx | HIV-1 |
| AF084936 | DRCBL | G | HIV-1 |
| AF193253 | VI1310 AF193253 | CRF05_DF | HIV-1 |
| AF190127 | VI991 | H | HIV-1 |
| AF193276 | KAL153-2 | CRF03_AB | HIV-1 |
| AF192135 | BW2117 | AJ | HIV-1 |
| AJ288982 | 95ML127 | CRF06_cpx | HIV-1 |
| AJ288981 | 97SE1078 | CRF06_cpx | HIV-1 |
| AJ271370 | YBF106 | N | HIV-1 |
| AJ237565 | 97NOGIL3 | ADHK | HIV-1 |

What we claim is:

1. A siRNA nucleic acid molecule which modulates expression of a nucleic acid molecule encoding HIV or a component of HIV.

2. An enzymatic nucleic acid molecule which modulates expression of a nucleic acid molecule encoding HIV or a component of HIV, wherein said enzymatic nucleic acid molecule is in an Inozyme, G-cleaver, Zinzyme or Amberzyme configuration.

3. An enzymatic nucleic acid molecule comprising a sequence selected from the group consisting of SEQ ID NOs. 77-139 and 149-158.

4. An enzymatic nucleic acid molecule comprising at least one binding arm wherein one or more of said binding arms comprises a sequence complementary to a sequence selected from the group consisting of SEQ ID NOs. 1-76 and 140-148.

5. A siRNA nucleic acid molecule comprising a sequence complementary to a sequence selected from the group consisting of SEQ ID NOs. 1-76 and 140-148.

6. The nucleic acid of any of claims 1-5, wherein said nucleic acid molecule is adapted to HIV infection or acquired immunodeficiency syndrome (AIDS).

7. The enzymatic nucleic acid molecule of any of claims 2-4, wherein said enzymatic nucleic acid molecule has an endonuclease activity to cleave RNA having a HIV sequence.

8. The enzymatic nucleic acid molecule of claim 2, wherein said enzymatic nucleic acid molecule is in an Inozyme configuration.

9. The enzymatic nucleic acid molecule of claim 2, wherein said enzymatic nucleic acid molecule is in a Zinzyme configuration.

10. The enzymatic nucleic acid molecule of claim 2, wherein said enzymatic nucleic acid molecule is in a G-cleaver configuration.

11. The enzymatic nucleic acid molecule of claim 2, wherein said enzymatic nucleic acid molecule is in an Amberzyme configuration.

12. The enzymatic nucleic acid molecule of claim 4, wherein said enzymatic nucleic acid molecule is in a DNAzyme configuration.

13. The enzymatic nucleic acid molecule of claim 4, wherein said enzymatic nucleic acid molecule is in a Hammerhead configuration.

14. The enzymatic nucleic acid molecule of claim 8, wherein said Inozyme comprises a sequence complementary to a sequence selected from the group consisting of SEQ ID NOs. 7-14.

15. The enzymatic nucleic acid molecule of claim 8, wherein said Inozyme comprises a sequence selected from the group consisting of SEQ ID NOs. 83-90.

16. The enzymatic nucleic acid molecule of claim 9, wherein said Zinzyme comprises a sequence complementary to a sequence selected from the group consisting of SEQ ID NOs. 15-22 and 145-148.

17. The enzymatic nucleic acid molecule of claim 9, wherein said Zinzyme comprises a sequence selected from the group consisting of SEQ ID NOs. 91-98 and 154-158.

18. The enzymatic nucleic acid molecule of claim 11, wherein said Amberzyme comprises a sequence complementary to a sequence selected from the group consisting of SEQ ID NOs. 15-47.

19. The enzymatic nucleic acid molecule of claim 11, wherein said Amberzyme comprises a sequence selected from the group consisting of SEQ ID NOs. 112-139.

20. The enzymatic nucleic acid molecule of claim 12, wherein said DNAzyme comprises a sequence complementary to a sequence selected from the group consisting of SEQ ID NOs. 15-27 and 140-144.

21. The enzymatic nucleic acid molecule of claim 12, wherein said DNAzyme comprises a sequence selected from the group consisting of SEQ ID NOs. 99-111 and 149-153.

22. The enzymatic nucleic acid molecule of claim 13, wherein said Hammerhead comprises a sequence complementary to a sequence selected from the group consisting of SEQ ID NOs. 1-6.

23. The enzymatic nucleic acid molecule of claim 13, wherein said Hammerhead comprises a sequence selected from the group consisting of SEQ ID NOs 77-82.

24. The nucleic acid molecule of any of claims 1-5, wherein said nucleic acid molecule comprises between 12 and 100 bases complementary to a nucleic acid molecule encoding HIV.

25. The nucleic acid molecule of any of claims 1-5, wherein said nucleic acid molecule comprises between 14 and 24 bases complementary to a nucleic acid molecule encoding HIV.

26. The nucleic acid molecule of any of claims 1-5, wherein said nucleic acid molecule is chemically synthesized.

27. The nucleic acid molecule of any of claims 1-5, wherein said nucleic acid molecule comprises at least one 2′-sugar modification.

28. The nucleic acid molecule of any of claims 1-5, wherein said nucleic acid molecule comprises at least one nucleic acid base modification.

29. The nucleic acid molecule of any of claims 1-5, wherein said nucleic acid molecule comprises at least one phosphate backbone modification.

30. A mammalian cell comprising the nucleic acid molecule of any of claims 1-5.

31. The mammalian cell of claim 30, wherein said mammalian cell is a human cell.

32. A method of reducing HIV activity in a cell, comprising contacting said cell with the nucleic acid molecule of any of claims 1-5, under conditions suitable for said reduction of HIV activity.

33. A method of treatment of a subject having a condition associated with the level of HIV, comprising contacting cells of said subject with the nucleic acid molecule of any of claims 1-5, under conditions suitable for said treatment.

34. The method of claim 32 further comprising the use of one or more drug therapies under conditions suitable for said treatment.

35. The method of claim 33 further comprising the use of one or more drug therapies under conditions suitable for said treatment.

36. A method of cleaving RNA of an HIV gene comprising contacting an enzymatic nucleic acid molecule of any of claims 2-4 with said RNA of HIV gene under conditions suitable for the cleavage.

37. The method of claim 36, wherein said cleavage is carried out in the presence of a divalent cation.

38. The method of claim 37, wherein said divalent cation is Mg²⁺.

39. The nucleic acid molecule of any of claims 1-5, wherein said nucleic acid molecule comprises a cap structure, wherein the cap structure is at the 5′-end, 3′-end, or both the 5′-end and the 3′-end of said nucleic acid molecule.

40. The nucleic acid molecule of claim 39, wherein the cap structure at the 5′-end, 3′-end, or both the 5′-end and the 3′-end comprises a 3′,3′-linked or 5′,5′-linked deoxyabasic ribose derivative.

41. An expression vector comprising a nucleic acid sequence encoding at least one nucleic acid molecule of any of claims 1-5 in a manner which allows expression of the nucleic acid molecule.

42. A mammalian cell including an expression vector of claim 41.

43. The mammalian cell of claim 42, wherein said mammalian cell is a human cell.

44. An expression vector comprising a nucleic acid sequence encoding at least one nucleic acid molecule of any of claims 3 or 4 in a manner which allows expression of the nucleic acid molecule, wherein said nucleic acid molecule is in a hammerhead configuration.

45. The expression vector of claim 41, wherein said expression vector further comprises a sequence for a nucleic acid molecule complementary to the RNA of HIV.

46. The expression vector of claim 41, wherein said expression vector comprises a nucleic acid sequence encoding two or more of said nucleic acid molecules, which may be the same or different.

47. The expression vector of claim 46, wherein said expression vector further comprises a sequence encoding a siRNA nucleic acid molecule complementary to the RNA of HIV gene.

48. A method for treatment of acquired immunodeficiency syndrome (AIDS) or an AIDS related condition comprising administering to a subject the nucleic acid molecule of any of claims 1-5 under conditions suitable for said treatment.

49. The method of claim 48, wherein said AIDS related condition is Kaposi's sarcoma, lymphoma, cervical cancer, squamous cell carcinoma, cardiac myopathy, rheumatic disease, or opportunistic infection.

50. The method of claim 48, wherein said method further comprises administering to said subject one or more other therapies.

51. The nucleic acid molecule of claim 2 or claim 4, wherein said nucleic acid molecule comprises at least five ribose residues, at least ten 2′-O-methyl modifications, and a 3′-end modification.

52. The nucleic acid molecule of claim 51, wherein said nucleic acid molecule further comprises phosphorothioate linkages on at least three of the 5′ terminal nucleotides.

53. The nucleic acid molecule of claim 51, wherein said 3′-end modification is a 3′-3′ inverted abasic moiety.

54. The method of claim 34 wherein said other drug therapies are antiviral therapy, monoclonal antibody therapy, chemotherapy, radiation therapy, analgesic therapy, or anti-inflammatory therapy.

55. The method of claim 54, wherein said antiviral therapy is treatment with AZT, ddC, ddI, d4T, 3TC, Ribavirin, delvaridine, nevirapine, efravirenz, ritonavir, saquinivir, indinavir, amprenivir, nelfinavir, or lopinavir.

56. The method of claim 35 wherein said other drug therapies are antiviral therapy, monoclonal antibody therapy, chemotherapy, radiation therapy, analgesic therapy, or anti-inflammatory therapy.

57. The method of claim 56, wherein said antiviral therapy is treatment with AZT, ddC, ddI, d4T, 3TC, Ribavirin, delvaridine, nevirapine, efravirenz, ritonavir, saquinivir, indinavir, amprenivir, nelfinavir, or lopinavir.

58. The method of claim 50 wherein said other drug therapies are antiviral therapy, monoclonal antibody therapy, chemotherapy, radiation therapy, analgesic therapy, or anti-inflammatory therapy.

59. The method of claim 58, wherein said antiviral therapy is treatment with AZT, ddC, ddI, d4T, 3TC, Ribavirin, delvaridine, nevirapine, efravirenz, ritonavir, saquinivir, indinavir, amprenivir, nelfinavir, or lopinavir.

60. A composition comprising a nucleic acid molecule of any of claims 1-5 in a pharmaceutically acceptable carrier.

61. The nucleic acid molecule of claim 1 or 2, wherein said component of HIV is nef.

62. The nucleic acid molecule of claim 1 or 2, wherein said component of HIV is vif.

63. The nucleic acid molecule of claim 1 or 2, wherein said component of HIV is tat.

64. The nucleic acid molecule of claim 1 or 2, wherein said component of HIV is rev.

65. The nucleic acid molecule of claim 1 or 2, wherein said component of HIV is LTR.

66. The nucleic acid molecule of claim 65, wherein said LTR is the 3′-LTR.

67. The nucleic acid molecule of claim 65, wherein said LTR is the 5′-LTR.

68. A method of administering to a cell a nucleic acid molecule of any of claims 1-5 comprising contacting said cell with the nucleic acid molecule under conditions suitable for said administration.

69. The method of claim 68, wherein said cell is a mammalian cell.

70. The method of claim 68, wherein said cell is a human cell.

71. The method of claim 68, wherein said administration is in the presence of a delivery reagent.

72. The method of claim 71, wherein said delivery reagent is a lipid.

73. The method of claim 72, wherein said lipid is a cationic lipid.

74. The method of claim 72, wherein said lipid is a phospholipid.

75. The method of claim 71, wherein said delivery reagent is a liposome.
